SEQUENCE LISTING (81 sequences)

SEQ ID NO: 1 - protein, 13 aa, synthetic construct
CASSTWTQETQYF

SEQ ID NO: 2 - protein, 13 aa, synthetic construct
CASSPNPAGELFF

SEQ ID NO: 3 - protein, 18 aa, synthetic construct
CSARARRAGGPGGETQVF

SEQ ID NO: 4 - protein, 16 aa, synthetic construct
CASSPGQPRATGELFF

SEQ ID NO: 5 - protein, 15 aa, synthetic construct
CASSVSGGTNEKLFF

SEQ ID NO: 6 - protein, 16 aa, synthetic construct
CASSQDRAGGTDTQYF

SEQ ID NO: 7 - protein, 10 aa, synthetic construct
CASSFTEAFF

SEQ ID NO: 8 - protein, 15 aa, synthetic construct
CATLGAGSHPLGYTF

SEQ ID NO: 9 - protein, 13 aa, synthetic construct
CASSESWDTEAFF

SEQ ID NO: 10 - protein, 16 aa, synthetic construct
CASSADRGLAGNTIYF

SEQ ID NO: 11 - protein, 13 aa, synthetic construct
CASSLNRDSEAFF

SEQ ID NO: 12 - protein, 17 aa, synthetic construct
CASSQETGISRNTEAFF

SEQ ID NO: 13 - protein, 13 aa, synthetic construct
CASSLAGAYEQYF

SEQ ID NO: 14 - protein, 13 aa, synthetic construct
CASSLVGAYEQYF

SEQ ID NO: 15 - protein, 13 aa, synthetic construct
CASSLVSTGELFF

SEQ ID NO: 16 - protein, 13 aa, synthetic construct
CASTRAHPDTQVF

SEQ ID NO: 17 - protein, 15 aa, synthetic construct
CASTAGPYYHQPQHF

SEQ ID NO: 18 - protein, 13 aa, synthetic construct
CASSLDSLAEAFF

SEQ ID NO: 19 - protein, 14 aa, synthetic construct
CASSLDGRWEKLFF

SEQ ID NO: 20 - protein, 15 aa, synthetic construct
CASSLDSGSGTEAFF

SEQ ID NO: 21 - protein, 16 aa, synthetic construct
CASSLAEGGADYGYTF

SEQ ID NO: 22 - protein, 14 aa, synthetic construct
CASSFGRTNGELFF

SEQ ID NO: 23 - protein, 17 aa, synthetic construct
CATSRDPSGRAGETQYF

SEQ ID NO: 24 - protein, 20 aa, synthetic construct
CASSWMDGLTGRRTTDTQYF

SEQ ID NO: 25 - protein, 14 aa, synthetic construct
CASSTDKTGQPQHF

SEQ ID NO: 26 - protein, 13 aa, synthetic construct
CASILPGQSEAFF

SEQ ID NO: 27 - protein, 17 aa, synthetic construct
CASSLGDRGFSGNTIVF

SEQ ID NO: 28 - protein, 17 aa, synthetic construct
CATSDRDRGPSNQPQHF

SEQ ID NO: 29 - protein, 16 aa, synthetic construct
CSARDLLAGVQETQYF

SEQ ID NO: 30 - protein, 15 aa, synthetic construct
CASSSEASGIYEQYF

SEQ ID NO: 31 - protein, 13 aa, synthetic construct
CSVLPISSYEQYF

SEQ ID NO: 32 - protein, 14 aa, synthetic construct
CASSEPRSGNTIYF

SEQ ID NO: 33 - protein, 14 aa, synthetic construct
CASSISLGEGELFF

SEQ ID NO: 34 - protein, 12 aa, synthetic construct
CASSLLGDEQYF

SEQ ID NO: 35 - protein, 12 aa, synthetic construct
CASSLGGYEQYF

SEQ ID NO: 36 - protein, 12 aa, synthetic construct
CASRRGPDTQYF

SEQ ID NO: 37 - protein, 15 aa, synthetic construct
CASSSPVQGTGELFF

SEQ ID NO: 38 - protein, 14 aa, synthetic construct
CASSLRTGTDTQYF

SEQ ID NO: 39 - protein, 10 aa, synthetic construct
CARLTGELFF

SEQ ID NO: 40 - protein, 16 aa, synthetic construct
CASNQFSQDSNQPQHF

SEQ ID NO: 41 - protein, 14 aa, synthetic construct
CASRPGYRYNEQFF

SEQ ID NO: 42 - protein, 16 aa, synthetic construct
CASSQGQTGGTQKLFF

SEQ ID NO: 43 - protein, 15 aa, synthetic construct
CASRRLAGGGETQYF

SEQ ID NO: 44 - protein, 14 aa, synthetic construct
CASSFEQGAGEQYF

SEQ ID NO: 45 - protein, 11 aa, synthetic construct
CASGTYNEQFF

SEQ ID NO: 46 - protein, 14 aa, synthetic construct
CASSDPAGDNEQFF

SEQ ID NO: 47 - protein, 17 aa, synthetic construct
CASRLGGGTGFNQPQHF

SEQ ID NO: 48 - protein, 16 aa, synthetic construct
CASSFTGSPPNTEAFF

SEQ ID NO: 49 - protein, 12 aa, synthetic construct
CASSLDTYEQYF

SEQ ID NO: 50 - protein, 12 aa, synthetic construct
CASSPAGQGGFF

SEQ ID NO: 51 - protein, 15 aa, synthetic construct
CASEGLAGPYNEQFF

SEQ ID NO: 52 - protein, 15 aa, synthetic construct
CATSDTGDRRYEQYF

SEQ ID NO: 53 - protein, 12 aa, synthetic construct
CASSATGVGYTF

SEQ ID NO: 54 - protein, 16 aa, synthetic construct
CASSLISGTGLTEAFF

SEQ ID NO: 55 - protein, 15 aa, synthetic construct
CASSDPASGYNEQFF

SEQ ID NO: 56 - protein, 15 aa, synthetic construct
CASSRVGRPHNEQFF

SEQ ID NO: 57 - protein, 12 aa, synthetic construct
CASSSSYNEQFF

SEQ ID NO: 58 - protein, 17 aa, synthetic construct
CASSQVNMGLTSEKLFF

SEQ ID NO: 59 - protein, 15 aa, synthetic construct
CASSLQAGSTDTQYF

SEQ ID NO: 60 - protein, 16 aa, synthetic construct
CSVRGGAGLPSGDQYF

SEQ ID NO: 61 - protein, 12 aa, synthetic construct
CSWVPPWNEQFF

SEQ ID NO: 62 - protein, 16 aa, synthetic construct
CASSQGWSGGFHEQYF

SEQ ID NO: 63 - protein, 20 aa, synthetic construct
CASSLTNTGTPSSYNSPLHF

SEQ ID NO: 64 - protein, 14 aa, synthetic construct
CASSYIALDEKLFF

SEQ ID NO: 65 - protein, 20 aa, synthetic construct
CASSLEIPGGTGHSTDTQYF

SEQ ID NO: 66 - protein, 13 aa, synthetic construct
CASSSGEIRENTF

SEQ ID NO: 67 - protein, 16 aa, synthetic construct
CASSLNLFQPRSEQFF

SEQ ID NO: 68 - protein, 15 aa, synthetic construct
CASSQQGAVSTEAFF

SEQ ID NO: 69 - protein, 17 aa, synthetic construct
CASSPNWQDPDGTEAFF

SEQ ID NO: 70 - protein, 14 aa, synthetic construct
CASRGTGWPGELFF

SEQ ID NO: 71 - protein, 18 aa, synthetic construct
CATSDPLWAGGLTDTQYF

SEQ ID NO: 72 - protein, 15 aa, synthetic construct
CASSGQGILGYGYTF

SEQ ID NO: 73 - protein, 15 aa, synthetic construct
CASSESGWSNQPQHF

SEQ ID NO: 74 - protein, 15 aa, synthetic construct
CASSPGQGFSYEQYF

SEQ ID NO: 75 - protein, 12 aa, synthetic construct
CATRTGTYEQYF

SEQ ID NO: 76 - protein, 13 aa, synthetic construct
CASSPREYQPQHF

SEQ ID NO: 77 - protein, 12 aa, synthetic construct
CASSIRGGELFF

SEQ ID NO: 78 - protein, 17 aa, synthetic construct
CASRGGGKPRGEYEQYF

SEQ ID NO: 79 - protein, 14 aa, synthetic construct
CSAGGGQGTFEQFF

SEQ ID NO: 80 - protein, 15 aa, synthetic construct
CASSLAGRRNEKLFF

SEQ ID NO: 81 - DNA, 2373 bp, synthetic construct
  source 1..2373: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagaggtcagcattcccgaaagcacaacctatacccctgcccagacctccaccctcgtgtccagctccaccaaagaggatgccgtccagacaccccctagaaaaagagctagaggagtgcaacagtccccctgtaacgctctgtgtgtggctcacattggccctgtggatagcggaaaccataacctcatcacaaacaatcacgatcagcatcagaggagaaataactccaactccagcgctacccctatcgtccagtttcagggagagtccaactgtctgaaatgctttagatatagactcaacgatagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctccattgctatgagcaactggaagactccagcgaagacgaagtggatatggcagacgattctgcactgcataaaaagtacccattcctgaacctgctgcataccccaccgcatcgcccaccgccactgtgtccgcaagctccacgcaagacccaatgcaagcgccgtctgggtaacgagcacgaggaatccaactccccgctggctactccgtgtacagccgaaatctatgcctatgcctataagaatctgaaagtggtccaatgcacagagacagacattagagaagtgcaacagctcctgctcggcacactgaatatcgtctgccctatctgtgcccctaagacaacagccgaaatctatagctatgcctataagcaactgaaagtgctccaatgcaagcgccgtctgggtaacgagcacgaggaatccaactccccgctggctactccgtgtgtttggccgactctggacccgtggaccgtggaaactaccacttcttccctgactatcactacctccaccaaggacggcaccactgttactgttcaactgcgtaccgctgagatttacgcttacgcttacaaaaacctcaaggtcgtgatgcacggcaggcacgtcaccctcaaggatatcgtcctggatctgcaaccccctgaccctgtgggactgcattgctatgagcaactggtggattccagcgaagacgaagtggatgaggtggacggacaggatagccaacccctcaagcaacactttcagattgtgacatgctgtcactgttacgaacagctggaggatagctccgaggatgaggtggacatggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacattctgacaaaggctagatttatcaaactgaattgcacaagaaaaggcaggtgcctccactgttggacaacctgtatggaagacatgctgcct

(19) United States
(12) Patent Application Publication
(10) Pub. No.: US 2026/0345782 A1
(43) Pub. Date: Nov. 5, 2026

(54) T CELL RECEPTORS GENERATED AS A RESULT OF HPV VACCINE THERAPY AND METHODS OF TREATING PATIENTS WITH SAME

(71) Applicants: PRECIGEN, INC., Germantown, MD (US); THE UNITED STATES OF AMERICA as represented by the Secretary, Department of Health and Human, Bethesda, MD (US)

(72) Inventors: Douglas E. BROUGH, Germantown, MD (US); Clint ALLEN, Bethesda, MD (US); Damodar R. ETTYREDDY, Germantown, MD (US); Qi YANG, Germantown, MD (US); Chen WANG, Germantown, MD (US)

(73) Assignees: PRECIGEN, INC., Germantown, MD (US); THE UNITED STATES OF AMERICA as represented by the Secretary, Department of Health and Human, Bethesda, MD (US)

(21) Appl. No.: 19/486,754

(22) PCT Filed: May 21, 2024
(86) PCT No.: PCT/US2024/030357

§ 371 Date: Nov. 21, 2025

Related U.S. Application Data
(60) Provisional application No. 63/503,578, filed on May 22, 2023.

(51) Int. Cl. A61K 40/46; A61K 40/11; A61K 40/32; A61K 47/64; A61K 51/08; A61P 35/00; C07K 14/705; C12N 5/0783

(52) U.S. Cl. CPC A61K 40/46; A61K 40/11; A61K 40/32; A61K 47/6425; A61K 51/08; A61P 35/00; C07K 14/7051; C12N 5/0636

(57) ABSTRACT

T cell receptors (TCRs) generated as a result of HPV vaccine therapy, including TCRs generated from any known HPV vaccines (e.g., HPV6/11 vaccines), engineered cells comprising such TCRs, and methods of treating patients with the same.

### STATEMENT REGARDING FEDERALLY SPONSORED RESEARCH OR DEVELOPMENT

This invention was made with Government support under project number ZIA BC012131 by the National Institutes of Health, National Cancer Institute. The Government has certain rights in the invention.

### BACKGROUND OF THE INVENTION

Human papillomavirus (HPV) is a common sexually transmitted infection that can cause a range of cancers, including cervical cancer, anal cancer, and head and neck cancer.

The most common HPV types are the low-risk HPV-6 and HPV-11, which are responsible for 90% of genital warts and a disease known as recurrent respiratory papillonlatosis (RRP), in which tumors grow in the airway. HPV also plays a role in the development of no-melanoma skin cancer (NMISC), including cutaneous squamous cell carcinoma (SCC), among chronic lymphocytic leukenia (CLL) and blood and marrow transplant (BMT) patients. HPV-16 and HPV-18, in particular, account for the majority of head and neck cancers (HNSCCs) and cancers of the cervix, anus, vagina, vulva, penis, tongue base, larynx, and tonsil.

Current standard therapeutic options for HNSCCs include and incorporate surgery and radiotherapy with concurrent chemotherapy (e.g., cisplatin and/or cetuximab). Unfortunately, the post-treatment burden on the patient following such modalities can be significant and permanent and may include dysphagia, dysphonia, xerostomia, scarring and disfigurement, and trismus. However, HPV-associated precancerous lesions, such as those of the vulva, vagina, anus, penis, as well as genital warts, are typically treated using physical elimination by cryotherapy (i.e., using extreme cold to destroy tissue), chemical cauterization (i.e., using a chemical to destroy tissue), and laser or surgical removal. Notably, in such pre-cancerous lesions physical elimination alone is not very effective, since 20-30% or more cases recur, with lesions both at previously treated sites due to failure of the procedure to eliminate the HPV, and at new sites due to new infections. When this occurs, radiotherapy and chemotherapy are then used with relative success; however, about 50% of the HPV-associated cancer patients still die of the disease.

Indeed, vaccines against HPV (e.g., Gardasil, Gardasil 9, and Cervarix) have been developed and largely successful in preventing HPV infection and associated cancers. However, there is a need for effective treatments for individuals who have already been infected with HPV and developed associated cancers. Conventional cancer therapies, such as chemotherapy and radiation, are often associated with significant side effects and may not be effective in all cases. Furthermore, cancer cells can become resistant to these therapies, leading to relapse and progression of the disease. Clearly, new treatment strategies are urgently needed to control the burden of HPV-related cancer.

Recent advances in immunotherapy have led to the development of T cell receptor (TCR) therapies for cancer treatment. TCRs are specialized receptors on T cells that can recognize specific antigens on cancer cells and trigger an immune response against them. TCR therapy involves isolating T cells from a patient, engineering them to recognize cancer-specific antigens, and reintroducing them into the patient to target and eliminate cancer cells. The use of engineered TCR therapy provides several advantages. The patient's own T cells may be equipped with desired specificities and allow generation of sufficient numbers of T cells in a short period of time while avoiding T cell exhaustion. In some embodiments of the invention, as disclosed herein, TCRs may be transduced into immune effector cells such as central memory T cells or T cells with stem cell characteristics, which may ensure better persistence and function upon transfer. Preferably, TCRs are transduced into cytotoxic T cells (CD8+ T cells; CTLs). Such TCR-engineered T cells (TCR-T cells) can be infused into cancer patients, such as cancer patients rendered lymphopenic by chemotherapy or irradiation, allowing for efficient engraftment but inhibiting immune suppression. As disclosed herein, the present invention relates, at least in part, to immune cells which recombinantly express an artificial T cell receptor (TCR) that targets HPV antigens.

In the case of HPV-associated cancers, TCRs generated as a result of HPV vaccine therapy may be particularly effective in recognizing and targeting cancer cells. This is because HPV vaccines stimulate the immune system to generate T cells that recognize and respond to HPV antigens. By isolating and engineering these TCRs to recognize cancer-specific antigens, they can be used to treat cancer associated with HPV infection.

Therefore, the present invention relates to T cell receptors (TCRs) generated as a result of HPV vaccine therapy and their use in treating cancer associated with HPV infection. The TCRs can be generated from any known HPV vaccine and may be engineered to recognize specific antigens on cancer cells associated with HPV infection, such as E6 and E7 proteins of HPV. The TCRs can be introduced into a patient with cancer associated with HPV infection using adoptive cell transfer or genetic modification of T cells. The introduction of the engineered TCRs can result in a reduction in tumor size and improved overall survival of the patient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

The present invention relates in part to a T cell receptor (TCR) generated as a result of therapy with an HPV vaccine. In certain embodiments, the HPV vaccine is selected from: an HPV quadrivalent recombinant vaccine; an HPV 9-valent recombinant vaccine; or an HPV bivalent recombinant vaccine.

In certain embodiments, the HPV vaccine is an HPV bivalent recombinant vaccine, for example, one that comprise a nucleic acid sequence having at least 80%, 90%, 95%, 97%, 98%, or 99% sequence identity with SEQ ID NO: 81. In certain such embodiments, the HPV vaccine comprises the nucleic acid sequence of SEQ ID NO: 81 or a codon degenerate variant thereof. In certain such embodiments, the HPV vaccine comprises the nucleic acid sequence of SEQ ID NO: 81.

In certain embodiments, the HPV vaccine is Gardasil.

In certain embodiments, the HPV vaccine is Gardasil 9 or Cervavac.

In certain embodiments, the HPV vaccine is Cervarix, Cecolin, or Walrinvax.

In certain embodiments, the TCR comprises an amino acid sequence having at least 80%, 90%, 95%, 97%, 98%, or 99% sequence identity with any one of SEQ ID NOs: 1-80. In certain such embodiments, the TCR comprises an amino acid sequence of any one of SEQ ID NOs: 1-80 or a conservatively-substituted variant thereof. In certain such embodiments, the TCR comprises the amino acid sequence of any one of SEQ ID NOs: 1-80.

In certain embodiments, TCR recognizes specific antigens on cancer cells associated with HPV infection. In certain such embodiments, the specific antigens are E6 and E7 proteins of HPV.

In certain embodiments, the TCR further comprises a modification that enhances the TCR's efficacy against HPV infection.

In certain embodiments, the TCR further comprises a modification that enhances the TCR's specificity to an HPV epitope.

In certain embodiments, the TCR is linked to an effector molecule. In certain such embodiments, the effector molecule is selected from the group consisting of: a cytokine, a toxin, a radioisotope, a chemotherapeutic agent, an antibody, an antibody fragment, and an antibody-drug conjugate.

The present invention also relates in part to a method of treating a disease or disorder associated with HPV infection in a subject in need thereof, the method comprising introducing into the subject the TCR of the present invention.

In certain embodiments, the TCR is introduced into the patient using adoptive cell transfer.

In certain embodiments, the disease or disorder is a cancer. In certain such embodiments, the cancer is cervical cancer, anal cancer, or head and neck cancer.

In certain embodiments, the disease or disorder is Recurrent Respiratory Papillomatosis.

The present invention further relates in part to a use of the TCR of the present invention in the preparation of a medicament for the treatment of a disease or disorder associated with HPV infection.

The present invention additionally relates to a method of preparing a TCR-T cell for use in treating a disease or disorder associated with HPV infection in a subject that has been administered an HPV vaccine, the method comprising isolating T cells from the subject, stimulating the T cells with HPV antigens, and isolating the HPV-specific TCR-T cells, wherein the HPV vaccine is selected from: an HPV quadrivalent recombinant vaccine; an HPV 9-valent recombinant vaccine; an HPV bivalent recombinant vaccine. In certain embodiments, the HPV vaccine comprises a nucleic acid sequence having at least 80%, 90%, 95%, 97%, 98%, or 99% sequence identity with SEQ ID NO: 81. In certain such embodiments, the HPV vaccine comprises the nucleic acid sequence of SEQ ID NO: 81 or a codon degenerate variant thereof. In certain such embodiments, the HPV vaccine comprises the nucleic acid sequence of SEQ ID NO: 81.

The present invention additionally relates in part to an engineered or isolated cell comprising the TCR of the present invention. In certain embodiments, the cell is a mammalian cell. In certain such embodiments, the cell is a mammalian cell.

In certain embodiments, the engineered or isolated cell is an immune cell. In certain such embodiments, the cell is a T cell.

The present invention further relates to a nucleic acid molecule encoding the TCR of the present invention.

The present invention also further relates to a vector comprising the aforementioned nucleic acid. In certain embodiments, the vector is an adenoviral vector.

The present invention additionally relates to a pharmaceutical composition comprising the aforementioned engineered or isolated cell and a pharmaceutically acceptable excipient. In certain embodiments, the pharmaceutical composition is for use in the preparation of a medicament for use in treating a disease or disorder associated with HPV infection.

The present invention also additionally relates to a method for producing an engineered cell expressing a TCR comprising introducing a nucleic acid of the present invention encoding the TCR to a cell. In certain embodiments, the cell is a T cell.

The present invention further additionally relates to a kit comprising the TCR of the present invention, the engineered cell of the present invention, the nucleic acid molecule of the present invention, the vector of the present invention, or the pharmaceutical composition of the present invention, and instructions for use in treating a disease or disorder associated with HPV infection.

The present invention also further additionally relates to a method of treating a disease or disorder associated with HPV infection in a subject in need thereof, the method comprising administering to the subject the TCR of the present invention, the engineered cell of the present invention, the nucleic acid molecule of the present invention, the vector of the present invention, or the pharmaceutical composition of the present invention, in combination with a therapy selected from: chemotherapy, radiation therapy, or checkpoint inhibitor therapy.

A further aspect of the present invention is a method of detecting HPV infection in a subject, the method comprising contacting a sample from the subject with the TCR of the present invention and detecting binding of the TCR to the sample, wherein binding of the TCR to the sample indicates the presence of HPV infection in the subject.

A yet further aspect of the present invention is a method of manufacturing the engineered cell of any one of claims 43-47, the method comprising culturing a cell and introducing into the cell a nucleic acid encoding the TCR of the present invention under conditions sufficient for expression of the TCR in the cell.

### BRIEF DESCRIPTION OF FIGURES

FIG. 1A depicts a dot plot showing the log-transformed fold change in HPV-specific T cell responses from the peripheral blood 6 weeks after completing treatment with an HPV 6/11 vaccine compared to before. Each dot represents the log-transformed fold change in IFNγ concentration following peptide stimulation with an individual pool of HPV peptides encoded in the vaccine pools to which IFNγ responses were not detected in the pre- or post-treatment samples are not shown. N=14 patients; insufficient pre-treatment PBMC were available from patient 5. Responders are in blue and non-responders are in gold.

FIG. 1B depicts a dot plot summarizing changes in HPV-specific peripheral blood responses. Significance was determined with a Mann-Whitney two-tailed test.

FIG. 1C depicts bar plots showing the fraction of the post-treatment TCRβ repertoire represented by the top 10 CDR3 frequencies determined to be HPV-specific in the HPV 6/11 peptide stimulation FEST assay. The Simpson clonality index is shown above each bar plot. Top horizontal bars indicate treatment response. R, responder; NR, non-responder.

FIG. 1D depicts a dot plot showing the log-transformed fold change in the frequency of the top 10 HPV-specific CDR3 sequences (determined in the FEST assay) from the peripheral blood 6 weeks after completing treatment with a HPV6/11 vaccine compared to before treatment without peptide stimulation.

FIG. 1E depicts a dot plot showing the summarized changes in the top 10 HPV-specific CDR3 sequences. Significance was determined with an unpaired two-tailed t-test.

FIG. 1F depicts bar plots summarizing the fraction of the top 10 peripheral blood HPV-specific CDR3 sequences (determined in the FEST assay) detected in the post-treatment peripheral blood that were undetectable (emergent), detected at lower frequencies (expanded) or detected and higher frequencies (contracted) compared to the pre-treatment peripheral blood. Top horizontal bars indicate treatment response.

FIG. 1G depicts a dot plot showing the log 2 transformed fold change in HPV-specific papilloma infiltrating lymphocytes (PIL) after completing treatment with an HPV6/11 vaccine compared to before. Each dot represents the log-transformed fold change in IFNγ spot count following co-culture of PIL with antigen presenting cells loaded with an individual pool of HPV peptides encoded in PRGN-2012. Pools to which IFNγ responses were not detected in the pre- or post-treatment samples are not shown. N=9 patients; no post-treatment biopsies were available for patients 7, 10, 11 and 13, and patients 2 and 3 failed to established pre-treatment PIL cultures.

FIG. 1H depicts a dot plot summarizing changes in HPV-specific PIL responses. Significance was determined with a Mann-Whitney two-tailed test.

FIG. 1I depicts clinical endoscopy images showing the pre-treatment and 6-week timepoint laryngeal appearance for patient 5. The red arrows indicate the pre-treatment and 6-week biopsy locations from which PIL were generated for experiments shown in FIG. 1J and FIG. 1K.

FIG. 1J depicts representative IFNγ ELISpot wells showing IFNγ spots following stimulation with pool 2 peptides (IFNγ spot counts are inset) in the pre-treatment and 6-week timepoints as well as negative (DMSO alone) and positive (PMA/lonomycin) controls.

FIG. 1K depicts a bar plot showing IFNγ concentrations following co-culture of the 6 weeks PIL sample with antigen presenting cells loaded with individual peptides included in pool 2.

FIG. 2A depicts representative photomicrographs of T cell immunofluorescence in baseline papilloma biopsies collected from responders (top row) and non-responders (bottom row) where subjects were treated with a HPV6/11 vaccine.

FIG. 2B depicts dot plots show the density of CD8 T cells in the papilloma and stroma or responders and non-responders where subjects were treated with a HPV6/11 vaccine. Significance was determined with a Mann-Whitney two-tailed test.

FIG. 2C depicts box-and-whisker plots show papilloma cell normalized HPV gene transcript counts in responders and non-responders where subjects were treated with a HPV6/11 vaccine, determined from single-cell RNA-seq. Significance determined with a two-way INOVA.

FIG. 2D depicts box-and-whisker plots show cell normalized reactome IFNγ signaling scores in different cell types (x-axis) in responders and non-responders, determined from single-cell RNA-seq. Significance determined with a two-way INOVA.

FIG. 2E, heat maps show the row-normalized chemokine transcript counts in different cell types (y-axis). Bar plots on the right indicate mean expression. Top horizontal bars indicate treatment response to an HPV 6/11 vaccine. Significance of the difference between responders and non-responders was determined with a two-way INOVA.

FIG. 2F depicts a violin plot showing the CXCR3 transcript counts for CD8 and CD4 papilloma T cells, determined from single-cell RNA-seq. Significance was determined with a Mann-Whitney two-tailed test.

FIG. 2G depicts a violin plot showing the percentage of (total) cells positive for CXCL9 or CXCL10. Significance was determined with a Mann-Whitney two-tailed test. Representative photomicrographs of RNAscope immunofluorescence are shown.

FIG. 2H depicts a dot plot showing the expression of select T cell-related genes across T lymphocyte clusters identified with single-cell RNA-seq, sorted by fold change in cell numbers detected in responders and non-responders (responders/non-responders; lower bar graph) where subjects were treated with a HPV6/11 vaccine. T cells enriched in non-responders are in the left columns, T cells enriched in responders and in the right columns. Circle color corresponds to scaled average expression; circle size denotes fraction of cells with non-zero gene expression of corresponding gene. Top bar graph represents total cell number.

### DETAILED DESCRIPTION OF THE INVENTION

It is to be understood that the present disclosure is not limited to the particular embodiments described herein and as such can vary. Although various features of the disclosure can be described in the context of a single embodiment, the features can also be provided separately or in any suitable combination. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

### DEFINITIONS

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Use of the term “including” as well as other forms, such as “include,” “includes,” and “included,” is not limiting; i.e., “including” does not mean “limited to.”

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional possible components, elements, or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “isolated” and its grammatical equivalents as used herein refer to the removal of a nucleic acid, protein, polypeptide, cell, or other material from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Polynucleotide,” “oligonucleotide,” “polynucleotide construct,” “gene,” “gene construct,” “heterologous gene” and their grammatical equivalents as used herein refer to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein can be introduced into a cell by, for example, transfection, transformation, or transduction.

“Polynucleotides encoding polypeptides”, and like or similar terms and phrases as used herein, include any polynucleotides which can be used to express (i.e., encode) the polypeptides. Indeed, it is well-known and understood by those of ordinary skill in the art how to construct any number of different polynucleotide sequences which may encode the same polypeptide (i.e., a polypeptide of identical amino acid sequence, although encoded by different sequences of codons (nucleotide triplets)). For example, one may use varying codons within a standard genetic code to generate a variety of polynucleotide sequences encoding a particular polypeptide. Moreover, one may also generate a variety of polynucleotide sequences encoding a particular polypeptide via use of codon selection as guided and selected via use of the “codon-use frequency” observed in a given organism; such as, by way of example, but not limited to, a Homo sapiens codon-use frequency table. See, e.g., https://www.genscript.com/tools/codon-frequency-table.

As used herein, the term “codon degenerate variant” refers to a modified nucleic acid sequence that encodes the same amino acid sequence as the original sequence but differs in the specific nucleotides comprising the codons. The genetic code is degenerate, meaning that multiple codons can code for the same amino acid. For example, the amino acid leucine can be encoded by six different codons: CTG, CTT, CTC, CTA, TTG, and TTA. A codon degeneracy table, also known as a genetic code table or codon table, is a chart that provides information about the relationship between codons (sequences of three nucleotides) and the corresponding amino acids they encode. The table lists the 64 possible codons and indicates which amino acid each codon represents. Table 1 is an example of a codon degeneracy table:

TABLE 1
Codon Degeneracy Table
Nucleotide Position in Codon - Amino Acid Encoded

| First | Second nucleotide | | | | Third |
|---|---|---|---|---|---|
| nucleotide | U | C | A | G | nucleotide |
| U | UUU - Phe | UCU - Ser | UAU - Tyr | UGU - Cys | U |
| | UUC - Phe | UCC - Ser | UAC - Tyr | UGC - Cys | C |
| | UUA - Leu | UCA - Ser | UAA - * | UGA - * | A |
| | UUG - Leu | UCG - Ser | UAG - * | UGG - Trp | G |
| C | CUU - Leu | CCU - Pro | CAU - His | CGU - Arg | U |
| | CUC - Leu | CCC - Pro | CAC - His | CGC - Arg | C |
| | CUA - Leu | CCA - Pro | CAA - Gln | CGA - Arg | A |
| | CUG - Leu | CCG - Pro | CAG - Gln | CGG - Arg | G |
| A | AUU - Ile | ACU - Thr | AAU - Asn | AGU - Ser | U |
| | AUC - Ile | ACC - Thr | AAC - Asn | AGC - Ser | C |
| | AUA - Ile | ACA - Thr | AAA - Lys | AGA - Arg | A |
| | AUG - Met | ACG - Thr | AAG - Lys | AGG - Arg | G |
| G | GUU - Val | GCU - Ala | GAU - Asp | GGU - Gly | U |
| | GUC - Val | GCC - Ala | GAC - Asp | GGC - Gly | C |
| | GUA - Val | GCA - Ala | GAA - Glu | GGA - Gly | A |
| | GUG - Val | GCG - Ala | GAG - Glu | GGG - Gly | G |

(U indicating “Uracil” as incorporated into mRNA in place of T (Thymine) as found in DNA; both of which form complementary base-pairs with “A” (Adenine); “*” indicates stop codons.)

Furthermore, publicly available software resources are readily available for computer-generated “reverse-translation” (also known as, “back translation” of polypeptide sequences (i.e., converting polypeptide sequences into nucleotide sequences encoding same). See, e.g., Madeira, F., et al., Nucleic Acids Res, 47(Wl), W636-W641 (2019); Madeira, F., et al., Curr Protoc in Bioinformatics, 66(1):e74 (2019); Chojnacki, S, et al., Nucleic Acids Res. 2017 Jul. 3; 45(Wl):W550-W553 (2017); Athey, J., et al., BMC Bioinformatics 18:391 (2017).

A “codon degenerate variant” thus refers to a nucleic acid sequence that has been modified to contain different codons, while still maintaining the same amino acid sequence when translated. A codon degenerate variant may be utilized to optimize gene expression or enhance protein production. By modifying the codons within a nucleic acid sequence, it is possible to utilize codons that are more frequently used or preferred by the host organism's translational machinery. This can lead to increased efficiency in protein expression or improved compatibility with a specific host organism.

“Polypeptide,” “peptide,” “polypeptide construct,” and “peptide construct,” and their grammatical equivalents as used herein, refer to a polymer of amino acid residues. A “mature protein” is a protein which is full-length and which, optionally, includes glycosylation or other modifications typical for the protein in a given cellular environment. As disclosed herein, embodiments of the invention include HPV antigens/antigenic polypeptides, peptides, and mature proteins described herein and also polynucleotides (DNA or RNA) that encode the same. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine.

The term “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window,” as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J Mal. Biol., 48:443 (1970); by the search for similarity method of Pearson and Lipman, Proc. Nat. Acad Sci US.A., 85:2444 (1988); by computerized implementations of these algorithms (including, but not limited to CLUSTAL in the PC/Gene program by Intelligentics, Mountain View Calif, GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 98%, 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98%, and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

The term “functional fragment,” or its grammatical equivalents, is used herein to mean a portion, fragment, or segment of a biological molecule that retains the essential functional characteristics or activities of the original biological molecule. The term “functional variant,” or its grammatical equivalents, is used herein to mean a modified form of a biological molecule that retains the essential functional characteristics or activities of the original molecule while exhibiting some degree of variation. It includes a biological molecule that has been altered, such as through genetic engineering or mutagenesis techniques, to introduce specific changes while preserving the biological molecule's overall functionality. A functional variant may have one or more amino acid substitutions, insertions, or deletions compared to the original molecule, while still maintaining the desired biological activity or function.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

Unless otherwise stated, the term “TCR” should be understood to encompass full TCRs as well as antigen-binding portions or antigen-binding fragments thereof. In some embodiments, the TCR is an intact or full-length TCR, such as a TCR containing the a chain and β chain. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α (Vα) chain and variable β (Vβ) chain of a TCR, or antigen-binding fragments thereof sufficient to form a binding site for binding to a specific MHC-peptide complex.

“T helper cells” (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including THI, TH2, TH3, TH9, THI7, TH22 or TFH (T follicular helper cells), which secrete different cytokines to facilitate different types of immune responses. Signaling from the APCs directs T cells into particular subtypes.

“Cytotoxic T cells” (TC cells, or CTLs) or “cytotoxic T lymphocytes” destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-I 0, adenosine, and other molecules secreted by regulatory T cells, the CD8+ cells can be inactivated to an anergic state, which prevents autoimmune diseases.

“Memory T cells” are a subset of antigen-specific T cells that persist long-term after an infection has resolved. They quickly expand to large numbers of effector T cells upon re-exposure to their cognate antigen, thus providing the immune system with memory against past infections. Memory T cells comprise three subtypes: central memory T cells (TcM cells) and two types of effector memory T cells (TEM cells and TEMRA cells). Memory cells can be either CD4+ or CD8+. Memory T cells typically express the cell surface proteins CD45RO, CD45RA and/or CCR7.

“Regulatory T cells” (Treg cells), formerly known as suppressor T cells, play a role in the maintenance of immunological tolerance. Their major role is to shut down T cell-mediated immunity toward the end of an immune reaction and to suppress autoreactive T cells that escaped the process of negative selection in the thymus.

“Natural killer T cells” (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CDId. Once activated, these cells can perform functions ascribed to both T helper (TH) and cytotoxic T (TC) cells (i.e., cytokine production and release of cytolytic/cell killing molecules). They are also able to recognize and eliminate some tumor cells and cells infected with herpes viruses.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained. Exemplary conservative amino acid substitutions are shown in the following chart:

TABLE 2
Exemplary Conservative Amino Acid Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

An amino acid sequence that differs from a reference amino acid sequence by only conservative amino acid substitutions will be referred to herein as a “conservatively-substituted variant” of the reference sequence.

In some embodiments, the functional variants may comprise the amino acid sequence of the reference protein with at least one non-conservative amino acid substitution. The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein. Amino acid substitutability is discussed in more detail, for example, in L. Y. Yampolsky and A. Stoltzfus, “The Exchangeability of Amino acids in Proteins,” Genetics 2005 August; 170(4):1459-1472.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHI, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDRI, CDR2, and CDR3, form the “hypervariable region” of an antibody, which is responsible for antigen binding.

“Antibody-like molecules” can be for example proteins that are members of the Ig-superfamily which are able to selectively bind a partner. MHC molecules and T cell receptors are such molecules. In one embodiment, the antibody-like molecule is an TCR. In one embodiment, the TCR has been modified to increase its MHC binding affinity.

The terms “functional antibody fragment” and “functional fragment of an antibody,” or their grammatical equivalents, are used interchangeably herein to mean a portion, fragment, or segment of the antibody that retains the essential functional characteristics or activities of the original antibody. In one embodiment, that activity is the ability to specifically bind to an antigen. (See, generally, Holliger et al., Nat. Biotech., 23(9):1126-1129 (2005)). The functional antibody fragment may comprise, for example, one or more CDRs, the variable region (or portions thereof), the constant region (or portions thereof), or combinations thereof. Non-limiting examples of functional antibody fragments include: (i) a antigen-binding fragment (Fab), which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a variable fragment (“Fv”) consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Functional antibody fragments are known in the art and are described in more detail in, e.g., U.S. Pat. No. 8,603,950.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

The term “proliferative disease” as referred to herein refers to a unifying concept in which excessive proliferation of cells and/or turnover of cellular matrix contributes significantly to the pathogenesis of the disease, including cancer.

“Patient” or “subject” as used herein refers to a mammalian subject diagnosed with or suspected of having or developing a proliferative disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” is referred to herein as a patient diagnosed with or suspected of having a disease or disorder, for instance, but not restricted to human papilloma virus (HPV) infection.

“Administering” is referred to herein as providing one or more compositions described herein to a patient or a subject. By way of example and not limitation, composition administration, e.g., injection, can be performed by intravenous injection, subcutaneous injection, intradermal injection, intraperitoneal injection, or intramuscular injection. One or more such routes can be employed. Parenteral administration can be, for example, by bolus injection or by gradual perfusion over time. Alternatively, or concurrently, administration can be by the oral route. Additionally, administration can also be by surgical deposition, or positioning of a medical device. A pharmaceutical composition can comprise a composition of the invention as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment,” “treating,” or its grammatical equivalents refers to obtaining a desired pharmacologic and/or physiologic effect. In embodiments, the effect is therapeutic, i.e., the effect partially or completely cures a disease and/or adverse symptom or pathological manifestation attributable to the disease. To this end, the inventive method comprises administering a therapeutically effective amount of a composition of the invention expressing the inventive nucleic acid sequence, or a vector comprising the inventive nucleic acid sequences.

As used herein, a “treatment interval” refers to a treatment cycle, for example, a course of administration of a therapeutic agent that may be repeated, e.g., on a regular schedule. In some embodiments, a dosage regimen may have one or more periods of no administration of the therapeutic agent in between treatment intervals.

The terms “administered in combination,” “co-administration,” or “co-administering,” or “co-providing” as used herein means that two (or more) different treatments are delivered to the subject during the course of the subject's affliction with a disease or disorder, for example, the two or more treatments are delivered after the subject has been diagnosed with the disease or disorder and before the disease or disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments may be partially additive, wholly additive, or greater than additive. The delivery may be such that an effect of the first treatment delivered is still detectable when the second is delivered.

The term “debulking” as used herein refers to the reduction of as much of the volume (i.e., bulk) of a tumor without the intention of a complete eradication. “Debulking” is usually usually achieved by surgical removal.

In some embodiments of the present invention, a first treatment and a second treatment may be administered simultaneously (e.g., at the same time), in the same or in separate compositions, or sequentially. Sequential administration refers to administration of one treatment before (e.g., immediately before; less than 5, 10, 15, 30, 45, or 60 minutes before; 1, 2, 3, 4, 6, 8, 10, 12, 16, 20, 24, 48, 72, 96 or more hours before; 4, 5, 6, 7, 8, 9 or more days before; or 1, 2, 3, 4, 5, 6, 7, 8 or more weeks before) administration of an additional (e.g., secondary) treatment. The order of administration of the first and secondary treatment may also be reversed.

The term “therapeutically effective amount,” “therapeutic amount,” “immunologically effective amount,” “anti-tumor effective amount,” “tumor-inhibiting effective amount” or its grammatical equivalents refers to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects.

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease or prevention of manifestation of a target pathology).

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### T Cell Receptors (TCRs)

T cell receptors (TCRs) are protein molecules found on the surface of T cells, which are a type of white blood cell involved in the adaptive immune response. TCRs are responsible for recognizing and binding to specific antigens presented on the surface of Antigen Presenting Cells (APCs) or any nucleated cell (e.g., all human cells in the body except erythrocytes), such as peptides derived from pathogens or abnormal cells. A TCR's variable domain contains the highly polymorphic loops referred to as complementarity determining regions (CDRs), which are responsible for binding to the peptide-presenting MHC. There are two major forms of TCRs: αβ TCR and γδ TCR. Both forms consist of two protein chains, known as alpha (α) and beta (β) chains for αβ TCRs and gamma (γ) and delta (δ) chains for γδ TCRs. These chains come together to form a heterodimeric structure.
1. αβ TCR: The majority of T cells in the human immune system express αβ TCRs. The α chain and β chain of αβ TCRs are encoded by separate gene segments, which undergo recombination during T cell development to generate diverse TCR specificities. The α and β chains each contain variable (V), diversity (D), and joining (J) gene segments, similar to the antibody gene rearrangement process. The combination of V, D, and J gene segments contributes to the unique antigen-binding specificity of the αβ TCR. The αβ TCR recognizes antigenic peptides presented in the context of major histocompatibility complex (MHC) molecules on the surface of antigen-presenting cells.
2. γδ TCR: In contrast to αβ TCRs, γδ TCRs are less prevalent in the immune system but still play important roles. The γ and δ chains of γδ TCRs are also encoded by separate gene segments and undergo recombination during T cell development. The γδ TCR gene rearrangement process is distinct from that of αβ TCRs. γδ T cells often exhibit a tissue-specific distribution and are found in epithelial tissues, such as the skin and gut. γδ TCRs can recognize a variety of antigens, including certain peptides and non-peptide molecules, independently of MHC presentation.

Both αβ TCRs and γδ TCRs participate in immune surveillance and response, but they have different functions and specificities. The αβ TCRs are predominantly involved in recognizing peptides presented by major histocompatibility complex (MHC) molecules, while γδ TCRs can have more diverse antigen recognition capabilities.

TCRs, and constructs encoding TCRs, that recognize MHC-antigen complexes, can be generated and introduced into T cells (known as TCR T cells), and the ensuing TCR-peptide-MHC interaction can be exploited to trigger an immune response. Greenbaum et al., Cancer Immunol Res 1 Nov. 2021; 9 (11): 1252-1261. There is an interest in using TCRs with higher than normal range of affinity for peptide-MHC antigens (type I), referred to as high affinity TCRs, to: 1) driving the activity of CD4 helper T cells (which do not have a CD8 co-receptor), or 2) developing soluble TCRs that can be used to directly target cells by attaching “effector” molecules (e.g., antibody Fc regions, toxic drugs, or antibody scFvs such as anti-CD 3 antibodies to form bispecific proteins) (Ashfield and Jakobsen, IDrugs, 9,554-9 (2006); Foote and Eisen Proc Natl Acad Sci USA, 97, 10679-81 (2000); Holler et al., Proc Natl Acad Sci USA, 97, 5387-92 (2000); Molloy et al., Curr Opin Pharmacol, 5,438-43 (2005); Richman and Kranz, Biomol Eng, 24,361-73 (2007)). This approach may also overcome the problem faced by some cancer patients whereby their T cells do not express TCRs with sufficient specificity and binding affinity for the underlying tumor antigen. For example, over 300 MHC restricted T cell defined tumor antigens (Cheever et al., Clin Cancer Res. 2009; 15(17):5323-5337) have been identified. These tumor antigens include mutated peptides, differentiation antigens, and over-expressed antigens, all of which serve as targets for therapy. Since most cancer antigens described to date are derived from intracellular proteins that can only be targeted at the cell surface in the context of MHC molecules, TCRs are ideal candidates for therapy as they have evolved to recognize this class of antigens.

Similarly, TCRs can detect peptides derived from viral proteins that have been naturally processed in infected cells and displayed on the cell surface by MHC molecules. However, patients with these diseases may not have an optimized TCR that binds and destroys infected cells. Finally, in methods with high specificity, TCRs may be used as receptor antagonists for autoimmune targets, or as delivery agents to immunosuppress local immune cell responses, thereby avoiding general immunosuppression.

The present invention provides TCRs, generated as a result of HPV vaccine therapy, which can be expressed in immune effector cells to enhance activity against specific targets (e.g., antitumor activity). In certain embodiments, T cells (e.g., cytotoxic T cells; CTLs) expressing such engineered TCRs are useful in the prevention and/or treatment of HPV infection, and/or cancer (e.g., a cancer expressing an HPV epitope), and/or precancerous lesions. In some embodiments, the TCR sequence (and the HPV epitope sequence to which it specifically binds) comprises at least one polypeptide sequence from the group consisting of SEQ ID NOs. 1-80 or a functional variant thereof (e.g., a polypeptide having at least 80%, 85%, 90%, 95%, 96%, 97%, 98%, or 99% sequence identity with any one of the amino acid sequences of SEQ ID NOs: 1-80 or a conservatively-substituted variant of any one of the amino acid sequences of SEQ ID NOs: 1-80).

The TCRs disclosed herein can be specific for HPV antigens, including HPV peptides E6 and E7. In certain embodiments, the TCRs are specific for antigens comprising at least one epitope having an amino acid sequence selected from the amino acid sequences set forth in WO 2022/115470 A1 or a functional variant thereof (e.g., an epitope having at least 80%, 85%, 90%, 95%, 96%, 97%, 98%, or 99% sequence identity with any one of the amino acid sequences disclosed in in WO 2022/115470 A1 or a conservatively-substituted variant of any one of the amino acid sequences disclosed in WO 2022/115470 A1).

The TCRs of the present invention can be generated from any known HPV, HPV6/11 vaccines, or the HPV vaccines disclosed in WO 2022/115470 A1. The TCRs can be isolated from T cells of patients who have received HPV vaccine therapy. The TCRs can be engineered to recognize specific antigens on cancer cells associated with HPV. Currently, as seen in the table below, three HPV vaccines are licensed for use by the U.S. Food and Drug Administration (“FDA”) in the United States:

TABLE 2
Characteristics of the three human papillomavirus (HPV)
vaccines licensed for use in the United States

| | | Quadrivalent | |
|---|---|---|---|
| Characteristic | Bivalent (2vHPV)* | (4vHPV)^{†} | 9-valent (9vHPV)^{§} |
| Brand name | Cervarix | Gardasil | Gardasil 9 |
| HPV-types | 16, 18 | 6, 11, 16, 18 | 6, 11, 16, 18, 31, 33, |
| | | | 45, 52, 58 |
| Manufacturer | GlaxoSmithKline | Merck and Co., Inc. | Merck and Co., Inc. |
| Manufacturing | Trichoplusia ni insect cell line | Saccharomyces | Saccharomyces |
| | infected with L1 encoding | cerevisiae (Baker's | cerevisiae (Baker's |
| | recombinant baculovirus | yeast), expressing L1 | yeast), expressing L1 |
| Adjuvant | 500 μg aluminum hydroxide, | 225 μg amorphous | 500 μg amorphous |
| | 50 μg 3-O-desacyl-4′ | aluminum | aluminum |
| | monophosphoryl lipid A | hydroxyphosphate | hydroxyphosphate |
| | | sulfate | sulfate |
| Volume per | 0.5 ml | 0.5 ml | 0.5 ml |
| dose | | | |
| Administration | Intramuscular | Intramuscular | Intramuscular |

See Petrosky et al., Use of 9-valent human papillomavirus (HPV) vaccine: updated HPV vaccination recommendations of the advisory committee on immunization practices. MMWR Morb Mortal Wkly Rep. 2015 Mar. 27; 64(11):300-4; see also FDA, Human Papillomavirus Vaccine https://www.fda.gov/vaccines-blood-biologics/human-papillomavirus-vaccine (last updated Jan. 31, 2018).

Cervarix, Gardasil, and Gardasil 9 are HPV vaccines presently licensed for use in the United States. However, numerous other HPV vaccines are known in the art, including at least Cecolin, Walrinvax, and Cervavac, which are licensed for use in countries other than the United States. Mo, Bi, Sheng et al., Hum Vaccin Immunother. 2022; 18(6):2092363; World Health Organization, Wkly Epidemiol Rec. 50 (97) (2022): 645-672; Chu, Bi, Huang et al., Lancet Reg. Health 2023; 100731.

Other HPV vaccines that may be used to generate TCRs of the present invention include at least those disclosed in the following publications: JP-7098330-B2; U.S. Ser. No. 10/772,947-B2; CN-104039833-B; U.S. Pat. No. 7,871,816-B2; CY-1107457-T1; US-2022152189-A1; DO-P2022000168-A; WO-2019151760-A1; EP-2386645-B1; WO-2017192418-A8; US-2009232842-A1; U.S. Ser. No. 10/125,175-B2; US-2009317415-A1; JP-7229151-B2; KR-20170032813-A; CA-2965498-A1; CN-114681603-A; ES-2519490-T3; RU-2356943-C2; CN-101765607-B; U.S. Pat. No. 7,371,390-B2; U.S. Pat. No. 8,187,606-B2; EP-3095798-B1; EP-1984388-B1; CA-2649555-A1; U.S. Pat. No. 8,263,560-B2; BR-PI0414845-A; KR-20090005011-A; BR-PI0909547-A2; BR-112013000031-A2; BR-PI0810959-B1; BR-PI0915076-A2; IS-7885-A; CN-101487009-A; CN-107080833-A; JP-2017507117-A; IL-178140-A; CN-102154325-A; BR-PI0508722-A; BR-112013030150-A2; NO-340633-B1; CN-101487010-A; EP-2940133-A4; BR-112017004181-A2; CN-104338126-B; HU-S1500061-I1; BR-PI0810951-B1; CN-109706144-A; EP-1758609-A1; BR-112012004928-B1; CN-112891526-A; CN-111529699-A; ZA-200504907-B; BR-112013000996-A2; CN-102008721-A; CN-110639013-A; CN-105597092-A; BR-112022009429-A2; WO-2011068934-A4; US-2010285058-A1; AR-054259-A1; U.S. Pat. No. 7,758,866-B2; CN-103992395-A; CN-102343103-A; CN-104998260-A; WO-2008145745-A1; CN-105363029-B; CN-108424926-A; RU-2509570-C2; CN-1900118-A; CN-109200270-B; CN-103667319-B; AR-053715-A1; CN-213677887-U; JP-2023516904-A; TH-75524-B; CN-109381699-A; TW-202313658-A; TH-118552-A; GB-0920319-D0; CN-114594068-A; CN-113984947-A; BR-PI0610396-A2; PE-20061434-A1; WO-2022171681-A1; US-2023123584-A1; TH-118552-B; WO-2017179232-A1; WO-2023014853-A1; CN-111228477-A; CN-114594067-A; EP-1007551-B1; NZ-505108-A; NZ-535085-A; CN-115569189-A; CN-1656116-A; NZ-516725-A; TW-201729837-A; AT-354662-T; KR-20040030599-A; SI-1210112-T1; KR-100785397-B1; CN-114134165-B; CN-110680918-B; WO-2022244815-A1; TW-202307211-A; ZA-200808893-B; CN-1308036-C; CN-114958741-A; CZ-20001244-A3; CN-111440811-A; AU-2002310802-A1; EP-3037103-A1; CY-1115704-T1; BR-PI0017420-B1; and BR-PI0610032-A2.

In one embodiment, the TCRs are generated from an HPV quadrivalent (Types 6, 11, 16, 18) recombinant vaccine (e.g., Gardasil). The TCRs can be isolated from T cells of patients who have received said vaccine. The TCRs can be engineered to recognize specific antigens on cancer cells associated with HPV infection, such as the E6 and E7 proteins. The engineered TCRs can then be introduced into the patient to treat cancer.

In one embodiment, the TCRs are generated from an HPV 9-valent recombinant vaccine (e.g., Gardasil 9). The TCRs can be isolated from T cells of patients who have received said vaccine. The TCRs can be engineered to recognize specific antigens on cancer cells associated with HPV infection, such as the E6 and E7 proteins. The engineered TCRs can then be introduced into the patient to treat cancer.

In another embodiment, the TCRs are generated from an HPV bivalent (Types 16 and 18) recombinant vaccine (e.g., Cervarix). The TCRs can be isolated from T cells of patients who have received said vaccine. The TCRs can be engineered to recognize specific antigens on cancer cells associated with HPV infection, such as the E6 and E7 proteins. The engineered TCRs can then be introduced into the patient to treat cancer.

In yet another embodiment, the TCRs are generated from the HPV vaccines disclosed in WO 2022/115470 A1. The TCRs can be isolated from T cells of patients who have received the HPV vaccines disclosed in the patent application. The TCRs can be engineered to recognize specific antigens on cancer cells associated with HPV infection, such as the E6 and E7 proteins. The engineered TCRs can then be introduced into the patient to treat cancer.

In another embodiment, the TCRs generated from the HPV vaccines disclosed in WO 2022/115470 A1 have any one of the amino acid sequences from the group of SEQ ID NOs. 1-80. In certain embodiments, the TCRs comprise an amino acid sequence having at least 90% sequence identity with any one of the amino acid sequences of SEQ ID NOs. 1-80 or a functional variant thereof (e.g., a polypeptide having at least 80%, 85%, 90%, 95%, 96%, 97%, 98%, or 99% sequence identity with any one of the amino acid sequences of SEQ ID NOs: 1-80 or a conservatively-substituted variant of any one of the amino acid sequences of SEQ ID NOs: 1-80).

The TCRs can be introduced into the patient using any known method of T cell therapy, such as adoptive cell transfer or genetic modification of T cells.

Provided herein is a method for producing a cell of any of the provided embodiments, comprising introducing any of the provided vectors into a cell in vitro or ex vivo. In certain embodiments, the vector is a viral vector and the introducing is carried out by transduction.

Provided herein is a composition comprising any of the engineered cells described herein. In some of any such embodiments, the engineered cells comprise CD4+ and/or CD8+ T cells. In certain embodiments, the engineered cells comprise CD4+ and CD8+ T cells.

Also provided herein is a composition, comprising an engineered CD8+ cell described herein and an engineered CD4+ cell described herein. In particular embodiments, the TCR binds to or recognizes a peptide epitope of HPV 6/11 in the context of an MHC molecule that is at least partially CD8-independent. In some of any such embodiments, the CD8+ cell and CD4+ cell are engineered with the same TCR and/or are each engineered with a TCR that binds to or recognizes the same peptide epitope of HPV 6/11 in the context of an MHC molecule. In certain embodiments, the composition further comprises a pharmaceutically acceptable excipient.

Provided herein is a method of treatment, comprising administering any of the provided engineered cells to a subject having a disease or disorder associated with HPV. Also provided herein is a method of treatment, comprising administering any of the provided composition to a subject having a disease or disorder associated with HPV. In particular embodiment, the disease or disorder is associated with HPV 6/11. In particular embodiments, the disease or disorder is cancer.

Provided herein is a method of treatment, comprising administering the engineered cell described herein to a subject having a disease or disorder associated with HPV. Also provided herein is a method of treatment, comprising administering a composition described herein to a subject having a cancer. Provided herein is one of any of a composition described herein for use in treating a disease or disorder associated with HPV in a subject.

Provided herein is a use of a composition of for the manufacture of a medicament for treating a disease or disorder associated with HPV in a subject. In some of any such embodiments, the disease or disorder is associated with HPV6/11. In particular embodiments, the disease or disorder is cancer. Also provided herein is a composition described herein for use in treating a cancer in a subject. Also provided herein is a use of any of a composition described herein for the manufacture of a medicament for treating a cancer in a subject. In further such embodiments, the cancer is associated with HPV6/11 infection. In particular embodiments, the subject is a human.

Nucleic Acids and Vectors Delivering and/or Expressing TCRs

Also disclosed are polynucleotides and polynucleotide vectors encoding the disclosed HPV antigen-specific TCRs that allow the expression of the HPV antigen-specific TCRs in the disclosed immune effector cells (e.g., T cells). In certain aspects of the invention, provided herein are isolated nucleic acids comprising a nucleotide sequence encoding a peptide that comprises an amino acid sequence from the group consisting of SEQ ID NOs. 1-80 or a functional variant thereof (e.g., a polypeptide having at least 80%, 85%, 90%, 95%, 96%, 97%, 98%, or 99% sequence identity with any one of the amino acid sequences of SEQ ID NOs: 1-80 or a conservatively-substituted variant of any one of the amino acid sequences of SEQ ID NOs: 1-80).

The term “suitable for transformation of host cells” means that the recombinant expression vector is a regulatory sequence selected based on the nucleic acid molecule of the present invention and a host cell to be used for expression, operably linked to the nucleic acid molecule. The terms “operably linked” or “operably linked” are used interchangeably, and are intended to mean that the nucleic acids are linked to regulatory sequences in a manner that allows expression of the nucleic acids.

Accordingly, the present invention provides a recombinant expression vector comprising a nucleic acid encoding an HPV-specific TCR, and regulatory sequences essential for transcription and translation of the inserted protein sequence. Suitable regulatory sequences can be derived from a variety of sources, including bacterial, fungal or viral genes. In general, vectors encoding TCRs used to transfect a subject's T cells should usually be non-replicating in the subject's T cells. A number of vectors based on viruses are known, wherein the number of copies of the virus retained in the cell is low enough to maintain the viability of the cell. Exemplary vectors include AdenoVerse™ vectors and gorilla adenovectors, as well as vectors based on HIV, SV40, EBV, HSV, BPV.

The selection of an appropriate regulatory sequence generally depends on the host cell selected and can be readily made by one skilled in the art. Examples of such regulatory sequences include transcription promoters and enhancer or RNA polymerase binding sequences, ribosome binding sequences including translation initiation sequences. Additionally, other sequences, such as origins of replication, additional DNA restriction sites, enhancers, and sequences conferring transcription inducibility, may also be included in the expression vector, depending on the host cell selected and the vector used. It will also be understood that essential regulatory sequences may be provided by the native protein and/or its contiguous regions.

The disclosed nucleic acids can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Alternatively, viral vectors (e.g., retroviral vectors, adenoviral vectors, adeno-associated viral vectors or lentiviral vectors) can be used to introduce the TCR constructs into T cells. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Preferably, gene transfer is into mammalian cells (e.g., PBMCs).

One example of a suitable promoter is the immediate early cytomegalovirus (CMV) promoter sequence. This promoter sequence is a strong constitutive promoter sequence capable of driving high levels of expression of any polynucleotide sequence operatively linked thereto.

Another example of a suitable promoter is Elongation Growth Factor-la (EF-la; EFla). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a TCR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes. Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g, mammalian, bacterial, yeast, or insect cell by any method in the art to produce transformed or transfected host cells. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g, human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells Comprising TCRs

Most preferably, the host cells engineered to express all or part of the disclosed TCRs of the invention include immune cells (e.g., immune effector cells). Such cells may be obtained from the subject (i.e., the donor) to be treated (i.e., autologous cells). However, in some embodiments, immune cell lines or donor cells other than the subject's own cells (i.e., allogeneic cells) are used.

Immune effector cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. Immune effector cells can be obtained from blood collected from a subject and/or donor using any number of techniques known to the skilled artisan, such as Ficoll™ separation. For example, cells from the circulating blood of an individual may be obtained by apheresis.

In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise any subset of T cells (e.g., T helper cells, cytotoxic T cells, regulatory T cells, memory T cells, natural killer T cells).

### TCR T Cells

Provided herein are methods for prophylaxis and treatment of HPV-associated diseases and cancers by the adoptive transfer of autologous or allogeneic HPV-specific, TCR-expressing cells (e.g., TCR T cells described herein). Such methods may include the generation of and/or the use of T cells (e.g., CTLs, CD8⁺ T cells, and/or CD4⁺ T cells) comprising the TCRs of the present invention.

The generation of peptide-specific T cells is known in the art and may include, for example, incubating a sample comprising T cells (e.g., a PBMC sample, an enriched sample, or a sample of isolated T cells) with antigenic peptides (i.e., peptides comprising T cell epitopes) or with antigen-presenting cells (APCs) that present one or more of such T cell epitopes (e.g., APCs that present a peptide comprising a CTL HPV epitope on a class I MHC complex), thereby inducing the sensitization (e.g., activation and proliferation) of peptide-specific T cells. For purposes of the present invention, such antigenic peptides and epitopes are HPV-specific.

In certain embodiments, a sample comprising CTLs (i.e., a PBMC sample) is incubated in culture with the antigenic HPV peptide (e.g., antigenic HPV6/11 peptides such as those disclosed in WO 2022/115470 A1) or with HPV antigen-presenting cells (APC) provided herein (e.g., “peptide-pulsed” cells that present said peptide, comprising an HPV epitope described herein, on a class I MHC complex).

In some embodiments, the APCs are autologous to the subject from whom the T cells were obtained. In some embodiments, the sample containing T cells is incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference. In some embodiments, the antigens are HPV antigens (e.g., E6 and E7)

In certain embodiments, the allelic HLA restriction (i.e., restriction to a specific HLA-A, HLA-B, or HLA-C allele) of such TCR-T cells is known. In some embodiments, the T cells used for generating the TCR-T cells of the invention are peptide-specific (i.e., sensitized to an antigenic peptide such as a viral peptide). In some embodiments, the T cells used for generating the TCR-T cells of the invention are polyfunctional T cells, i.e., those T cells that are capable of inducing multiple immune effector functions, that provide a more effective immune response to a pathogen than do cells that produce, for example, only a single immune effector (e.g. a single biomarker such as a cytokine or CD 107a). Less-polyfunctional, monofunctional, or even “exhausted” T cells may dominate immune responses during chronic infections, thus negatively impacting protection against virus-associated complications. In further preferred embodiments, the TCR-T cells of the invention are polyfunctional. In certain embodiments, at least 50% of the T cells used for generating the TCR-T cells of the invention are CD4⁺ T cells. In some such embodiments, said T cells are less than 50% CD4⁺ T cells. In still further embodiments, said T cells are predominantly CD4⁺ T cells. In some embodiments, at least 50% of the T cells used for generating the TCR-T cells of the invention are CD8⁺ T cells. In some such embodiments, said T cells are less than 50% CD8⁺ T cells. In still further embodiments, said T cells are predominantly CD8⁺ T cells. In some embodiments, the T cells (e.g., the donor samples, the sensitized T cells, and/or TCR-T cells described herein) are stored in a cell library or bank before they are administered to the subject.

In some embodiments, the engineered TCR-T cells expressing the disclosed TCRs further express a dominant-negative mutation that effects immune checkpoint blockade (e.g., express a dominant-negative form of an immune checkpoint molecule such as PD-1). Without intending to be an exhaustive list, the immune checkpoint molecule is selected from programmed death 1 (PD-1), cytotoxic T lymphocyte antigen-4 (CTLA-4), B- and T-lymphocyte attenuator (BTLA), T cell immunoglobulin mucin-3 (HM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD 160. The immune checkpoint molecule may also be transforming growth factor β (TGF-β) receptor. In certain preferred embodiments, the immune checkpoint molecule is CTLA-4. In certain embodiments, the immune checkpoint molecule is PD-1.

In some embodiments, in one or more T cell populations, cells that are positive for a specific marker, such as a surface marker or negative for the specific marker, are enriched or depleted. In some cases, these markers are markers that are absent or are expressed at relatively low levels in certain T cell populations (e.g., non-memory cells), but are present or expressed at relatively high levels in other specific T cell populations (e.g., memory cells).

T cells of the present invention may be isolated by any isolation method known in the art, or a combination thereof. Isolation methods may vary depending on the desired purity, cell yield, and downstream applications. Researchers and clinicians may choose the most suitable technique based on the specific requirements of their study or clinical procedure.

In certain embodiments, T cells may be isolated from a patient or subject's cell sample by density gradient centrifugation. Methods Mol Biol. 2019; 1930:11-17. In density gradient centrifugation, the patient or subject's cell sample is sample over a density gradient medium, typically a solution like Ficoll or Percoll. Centrifugation is then performed, causing the different cell types to separate based on their density. T cells, being relatively dense, will form a distinct layer in the gradient, allowing for their isolation.

In certain embodiments, T cells may be isolated from a patient or subject's cell sample by magnetic cell separation. Nanoscale, 2017; 9:13592-13599. In magnetic cell separation, T cells are be targeted using specific antibodies attached to magnetic beads. The cell sample is incubated with the antibody-coated beads, allowing the beads to bind to the T cells. The cell suspension is then passed through a magnetic field, causing the T cells (bound to the beads) to be retained while other cells are washed away. The T cells can then be released from the beads for further use.

In certain embodiments, T cells may be isolated from a patient or subject's cell sample by flow cytometry sorting. Cytometry A., 2019; 95(6):647-654. Fluorescently labeled antibodies specific to T cell surface markers (e.g., CD3, CD4, CD8) are used to identify and sort T cells. The cell sample is stained with the antibodies, and the labeled cells are then passed through a flow cytometer. The flow cytometer detects the labeled cells and separates them based on their fluorescence properties, allowing for the isolation of T cells.

In some embodiments, T cells are isolated from peripheral blood mononuclear cells (PBMC) samples by negative selection of markers, such as CD14, that are expressed on non-T cells, such as B cells, monocyte cells or other leukocyte cells. In some embodiments, CD4⁺ or CD8⁺ selection steps are used to isolate CD4⁺ helpers and CD8⁺ cytotoxic T cells. These CD4⁺ and CD8⁺ groups can be further classified into subgroups by positive or negative selection of markers expressed on one or more unsensitized, memory and/or effector T cell subgroups or to a relatively high degree.

In some embodiments, the T cells are autologous T cells. In one method, a tumor sample is obtained from a patient to obtain a single cell suspension. The single cell suspension can be accomplished by any suitable method, for example mechanically or enzymatically. Single cell suspensions of tumor enzyme lysates may be cultured in interleukin-2 (IL-2). Cells may be cultured until confluence (e.g., about 2×10⁶ lymphocytes) is reached, e.g., from about 5 days to about 21 days, preferably from about 10 days to about 14 days. For example, cells can be cultured for 5 days, 5.5 days or 5.8 days to 21 days, 21.5 days or 21.8 days, such as 10 days, 10.5 days or 10.8 days to 14 days, 14.5 days, or 14.8 days.

The cultured T cells can be collected and rapidly proliferated. Rapid proliferation increases the number of antigen-specific T cells of at least about 50 fold (e.g., 50 fold, 60 fold, 70 fold, 80 fold, 90 fold or 100 fold or more) over a period from about 10 days to about 14 days. More preferably, rapid proliferation is increased by at least about 200 times (e.g., 200 times, 300 times, 400 times, 500 times, 600 times, 700 times, 800 times, 900 times or more) for about 10 days to about 14 days.

T cells of the present invention may also be propagated by any of a variety of propagation methods as known in the art. For example, T cells using non-specific T cell receptor stimulation in the presence of feeder lymphocytes and interleukin-2 (IL-2) or interleukin-15 (IL-15) (IL-2 is preferred) can quickly multiply. T cells may also be rapidly proliferated by stimulating peripheral blood mononuclear cells (PBMCs) in vitro with one or more cancer antigens that can be expressed (including their antigenic moieties, such as epitope(s) or cells). The T cells induced in vitro may be rapidly proliferated by re-stimulation with the same cancer antigen(s) pulsed on antigen presenting cells expressing HLA-A2. Alternatively, the T cells may be re-stimulated with, for example, irradiated autologous lymphocytes or irradiated HLA-A2⁺ allogeneic lymphocytes and IL-2.

In other embodiments, the autologous T cells may be modified to express T cell growth factors that promote growth and activation of the autologous T cells. Suitable T cell growth factors include, for example, interleukin IL-2, IL-7, IL-15 and IL-12. Suitable methods of modification are known in the art. Sambrook et al., Molecular Cloning: A Laboratory Manual, 3^{rd} ed., Cold Spring Harbor Press, Cold Spring Harbor, NY 2001; Ausubel et al., Current Protocols in Molecular Biology, Greene Publishing Associates and John Wiley & Sons, NY, 1994. In certain embodiments, modified autologous T cells express T cell growth factors at high levels. The coding sequence of T cell growth factor coding sequence, such as IL-12, is readily available in the art as a promoter is such, and the operable binding of the promoter to the T cell growth factor coding sequence promotes high levels of expression.

In certain embodiments, T cell growth factors that promote the growth and activation of autologous T cells are administered to the subject simultaneously with or following the autologous T cells. The T cell growth factor can be any suitable growth factor that promotes growth and activation of autologous T cells. Examples of suitable T cell growth factors are interleukin (IL)-2, IL-7, IL-15 and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL-2. IL-12 is the preferred T cell growth factor.

T cells of the present invention may be engineered to express the novel T-cell receptors (TCRs) using various genetic engineering techniques known in the art. Careful design and optimization techniques, quality control measures and extensive characterization of the engineered T cells, are crucial to ensure the proper expression and function of the TCRs and to ensure their safety and efficacy in clinical applications.

In certain embodiments, retroviral or lentiviral transduction is used to engineer T cells to express the desired TCRs. Viruses, 2021; 13(8):1528; Hum Gene Ther., 2009; 20(6):630-40. Accordingly, in such embodiments, a retroviral or lentiviral vector is used to deliver the TCR gene into the T cells, and the vector comprises a nucleic acid sequence encoding the desired TCR, along with nucleic acid sequences encoding other necessary elements such as promoters and selection markers. In such embodiments, the T cells are isolated from the patient and activated to make them receptive to gene transfer. The vector carrying the TCR gene is then introduced into the activated T cells via transduction. The viral vector integrates the TCR gene into the T cells' genome, allowing the T cells to express the desired TCRs on their surfaces.

In certain embodiments, genome editing techniques like TALEN (Transcription Activator-Like Effector Nucleases) or CRISPR/Cas9 (Clustered Regularly Interspaced Short Palindromic Repeats/CRISPR-associated protein 9) are used to engineer T cells to express the desired TCRs. Gene Therapy, 2014; 21:539-548; Blood, 2018; 131(3):311-322. TALEN and CRISPR involve the targeted modification of the T-cell genome to insert the desired TCR gene or modify the endogenous TCR locus. TALENs or CRISPR/Cas9 are designed to recognize and cleave specific DNA sequences at the TCR gene loci. This enables the introduction of the desired TCR gene or disruption of the endogenous TCR gene, allowing for the expression of the engineered TCR.

In certain embodiments, the engineered T cells expressing the TCRs (i.e., TCR-T cells) of the present invention are expanded in culture to generate a sufficient number of cells for the therapeutic purposes or research applications disclosed herein.

### Therapeutic Methods

Immune effector cells expressing the disclosed TCRs can elicit a therapeutically beneficial immune response against HPV antigen-expressing cancer cells (e.g., HPV-associated cancers). For example, an anti-tumor immune response elicited by the disclosed TCR-modified immune effector cells may be an active or a passive immune response. In addition, the TCR-mediated immune response may be part of an adoptive immunotherapy approach in which TCR-modified immune effector cells induce an immune response specific to an HPV antigen.

Adoptive transfer of immune effector cells expressing engineered TCRs is a promising anti cancer therapy. Accordingly, in some aspects of the invention, provided herein are methods of treating a HPV-associated cancer in a subject, the method comprising administering an effective amount of an adoptive immunotherapy composition comprising the TCR-expressing cells contemplated herein. Following the collection of a patient's immune effector cells, the cells may be genetically engineered to express the disclosed HPV antigen-specific TCRs, thus tailoring the specific antigenicity of said immune effector cells (e.g., T cells) and infusing them back into the patient. Moreover, immune effector cells obtained from a donor other than the patient (i.e., allogeneic to the patient) may be genetically engineered to express the disclosed HPV antigen-specific TCRs, then the TCR-containing cells are infused into the patient. In certain specific embodiments, the immune effector cells which comprise an anti-HPV antigen TCR polypeptide are allogeneic HPV-specific cytotoxic T cells.

For example, a certain embodiment of the present invention are therapeutic compositions comprising the TCR-T cells provided herein. In some embodiments, such compositions are used to treat cancer and/or HPV infection in a subject by administering to the subject an effective amount of the composition. In some embodiments, the engineered TCR-T cells are not autologous to the subject. In some embodiments, the TCR-T cells are autologous to the subject. In some embodiments, the TCR-T cells are stored in a cell bank before they are administered to the subject. Therefore, in some embodiments, the disclosed immune effector cells that comprise one or more of the engineered TCR polypeptides of the present invention are allogeneic or autologous immune effector cells.

The disclosed TCR-modified immune effector cells (e.g., TCR-T cells) may be administered either alone, or as a pharmaceutical composition in combination with diluents and/or with other components such as IL-2, IL-15, or other cytokines or cell populations. Briefly, pharmaceutical compositions may comprise a targeting cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject).

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient by direct administration to an organ, subcutaneously, intradermally, intratumorally, intrathecally, intranodally, intramedullary, intramuscularly, intrapleurally, intracranially, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

The appropriate dosage of TCR therapy can be determined based on the type of disease to be treated, the severity and course of the disease, the subject's clinical condition, the subject's clinical history and response to treatment, and the attending physician's discretion.

In some embodiments, naked DNA encoding a TCR of the present invention or an appropriate vector comprising such a TCR can be introduced into a subject's T cells (e.g., T cells obtained from human patients with cancer caused by HPV-infection). Methods for stably transfecting T cells by electroporation using naked DNA are known in the art. See, e.g., U.S. Pat. No. 6,410,319.

When the engineered immune cells (e.g., TCR T-cells) are capable of expressing the desired level of a TCR of the present invention, such cells can be re-introduced or administered to the subject to activate the anti-tumor response in the subject.

To facilitate administration, the transduced cells (e.g., TCR T cells) may be made of a pharmaceutical composition or an implant suitable for in vivo administration with a suitable pharmaceutically acceptable carrier or diluent. Methods of making such compositions or implants are well known in the art. See, e.g., Remington's Pharmaceutical Sciences, 16th Ed., Mack, ed. (1980). Where appropriate, engineered cells expressing TCRs of the present invention (e.g., TCR T cells) may be formulated in semi-solid or liquid form preparations, such as capsules, solutions, injections, inhalants or aerosols, in the usual manner for their respective routes of administration.

Methods known in the art can be used to prevent or minimize the release and absorption of the composition until the composition reaches the target tissue or organ, or to ensure the sustained effectiveness of the composition. In general, it is desirable to use pharmaceutically acceptable forms that render cells expressing the TCR effective. Thus, preferably, the T cells may be prepared as a pharmaceutical composition containing a Hanks' equilibrium salt solution or an equilibrium salt solution such as conventional saline solution.

### Diagnostic Methods

In addition to the therapeutic applications of the TCRs described herein, the TCRs can also be used for diagnostic purposes, such as detecting HPV infection in a subject. HPV infection is a major cause of several types of cancer, including cervical, anal, and head and neck cancers. Early detection of HPV infection can facilitate timely intervention and treatment, potentially preventing the development of these cancers.

One method of detecting HPV infection using the TCRs of the present invention involves contacting a sample from a subject with the TCR and detecting binding of the TCR to the sample. The sample can be any biological sample that may contain HPV-infected cells, such as a tissue biopsy, a swab from a potentially infected area, or a blood sample. Binding of the TCR to the sample indicates the presence of HPV antigens and, thus, HPV infection in the subject. The detection of TCR binding can be performed using various assays known in the art, such as ELISA, flow cytometry, or immunohistochemistry.

In one embodiment, the TCR used for detecting HPV infection is linked to a detectable label, such as a fluorescent dye, an enzyme, or a radioisotope. The labeled TCR is then contacted with the sample, and the presence of the label is detected using an appropriate method, such as fluorescence microscopy, colorimetric assay, or autoradiography. In another embodiment, the TCR is unlabeled, and binding of the TCR to the sample is detected using a secondary reagent, such as a labeled antibody specific for the TCR. The diagnostic methods using the TCRs of the present invention can be performed in vitro or in vivo, depending on the specific requirements and constraints of the clinical setting.

### Pharmaceutical Preparation

In carefully selected embodiments, cells expressing a TCR of the present invention or polynucleotides encoding a TCR of the present invention, can be included in a vaccine composition, which is administered to a subject. It is contemplated that administration will induce a therapeutic immune response in the subject against cancer caused by HPV infection. Therapeutic compositions for pharmaceutical use in a subject can include TCR compositions such as soluble TCRs (optionally attached to imaging agents) and pharmaceutically acceptable carriers.

“Protective immune response” as used herein refers to the response of the mammalian host immune system to cancer. A protective immune response can provide a therapeutic effect for cancer treatment, such as reducing the size of the tumor or increasing the survival rate.

Proper formulation of the pharmaceutical composition is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). Formulation and dosage of the composition also depends upon factors like body weight, severity of disease, type of disease to be treated, existing or current therapeutic intervention, and idiopathic specificity of the patient.

The therapeutic compositions disclosed herein may be administered by intravenous, intradermal, intraarterial, intraperitoneal, intralesional, intracranial, intraarticular, intraprostatic, intrapleural, intratracheal, intranasal, intravitreal, intravaginal, intrarectal, intratumor, intramuscular, intraperitoneal, subcutaneous, subconjunctival, intravesical, intramucosal, pericardial, intranasal, intraocular, oral, topical, and by inhalation, injection, infusion, continuous infusion, lavage and local perfusion. The therapeutic composition may be administered to a subject via a catheter, as a cream, as a lipid composition, by ballistic particulate delivery, or by other methods or combinations thereof, as is known to those skilled in the art (see, e.g., Remington, The Science and Practice of Pharmacy, 21^{st} Ed. Lippincott Williams and Wilkins, 2005).

Any suitable carrier known to those skilled in the art can be used in the pharmaceutical composition of the present invention, but the type of carrier will vary depending on the method of administration. For parenteral administration, such as subcutaneous injection, the carrier preferably comprises water, saline, alcohol, fat, wax or buffer. For oral administration, any of the above carriers, or solid carriers such as mannitol, lactose, starch, magnesium stearate, sodium saccharinate, talc, cellulose, glucose, sucrose and magnesium carbonate can be used. Biodegradable microspheres (e.g., polylactic galactide) can also be used as carriers for the pharmaceutical compositions of the present invention. Suitable biodegradable microspheres are disclosed, for example, in U.S. Pat. Nos. 4,897,268 and 5,075,109.

Suitable pharmaceutical compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The pharmaceutical composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution.

In some embodiments, the vaccine composition can be administered by microstructured transdermal delivery or ballistic particulate delivery. Microstructures as carriers for vaccine formulations are well known in the art as preferred configurations for vaccine use (Gerstel and Place 1976 (U.S. Pat. No. 3,964,482); Ganderton and McAinsh 1974 (U.S. Pat. No. 3,814,097); U.S. Pat. Nos. 5,797,898, 5,770,219 and 5,783,208, and US patent application 2005/0065463). Such vaccine compositions formulated for ballistic particulate delivery can include isolated TCR peptides disclosed herein immobilized on the surface of a carrier substrate. In such embodiments, the carrier substrate may include, but is not limited to, microcapsules, microparticles, microspheres, nanocapsules, nanoparticles, nanospheres, or combinations thereof.

Microstructures or ballistic particulates that function as carrier substrates for the TCRs disclosed herein, such as soluble TCRs, can be comprised of biodegradable and non-biodegradable materials, such carrier substrates being synthetic polymers, silica, lipids, carbohydrates, proteins, lectins, Ionic agents, crosslinking agents, and other microstructural components that can be used in the art. Protocols and reagents for immobilizing the peptides of the present invention on a carrier substrate composed of these materials are widely available and available in the art.

In other embodiments, the vaccine composition comprises an immobilized or encapsulated TCR or soluble TCR disclosed herein, and a carrier substrate. In this embodiment, the carrier substrate is, but is not limited to, lipid microspheres, lipid nanoparticles, ethosomes, liposomes, niosomes, phospholipids, sphingosomes, surfactants, transferosomes, emulsions, or combinations thereof. The production and use of liposomes and other lipid nanocarriers and microcarrier formulations are generally known to those skilled in the art, and liposomes, microparticles, nanocapsules, and the like are widely used for the delivery of therapeutic agents (e.g., U.S. Pat. No. 5,741,516). Numerous methods have been reviewed for liposomes and liposome-like agents as potential drug carriers, including encapsulation of peptides (U.S. Pat. Nos. 5,567,434; 5,552,157; 5,565,213; 5,738,868 and 5,795,587)

In addition to the delivery methods described herein, numerous alternative techniques for administering the disclosed vaccine compositions are also contemplated. As a non-limiting example, the vaccine composition may include sonophoresis (i.e., ultrasound) that has been described and used in U.S. Pat. No. 5,656,016 to improve the rate and efficacy of drug penetration through the circulatory system (and into the circulatory system); It can be administered by intradermal injection (U.S. Pat. No. 5,779,708), or by feedback controlled delivery (U.S. Pat. No. 5,697,899).

Various optional adjuvants can be used in the vaccines of the present invention to improve the immune response non-specifically. Most adjuvants are substances designed to protect antigens from rapid catabolism, such as aluminum hydroxide or mineral oil, and lipid A, Bortella pertussis or Mycobacterium tuberculosis. Non-specific immune response stimulators. Suitable adjuvants are, for example, incomplete Freund's adjuvant and fully Freund's adjuvant [manufactured by Difco Laboratories, Detroit, MI] and Merck Adjuvant 65 [Raway, New Jersey, USA] Commercially available from Merck and Company, Inc.). Other suitable adjuvants include alum, biodegradable microspheres, monophosphoryl lipid A and quill A.

Soluble TCRs can be formulated in compositions in neutral or salt form. Pharmaceutically acceptable salts include acid addition salts (formed as free amino groups of proteins), which are formed, for example, from inorganic acids such as hydrochloric acid or phosphoric acid, or organic acids such as acetic acid, oxalic acid, tartaric acid, mandelic acid, and the like. Further, salts formed with free carboxyl groups include, for example, inorganic bases such as sodium hydroxide, potassium hydroxide, ammonium hydroxide, calcium hydroxide, or ferric hydroxide; And organic bases such as isopropylamine, trimethylamine, histidine, procaine, and the like.

In some embodiments, the composition may include various antioxidants to delay oxidation of one or more components. Additionally, but not limited to, the action of microorganisms by preservatives such as various antibacterial and antifungal agents including, but not limited to, parabens (e.g. methylparaben, propylparaben), chlorobutanol, phenol, sorbic acid, thimerosal, or combinations thereof.

If necessary, the TCRs of the present invention may be included in a suitable amount in a suitable solvent together with various other ingredients listed above, and then filtered and sterilized to prepare a sterile injection solution. Generally, dispersions are prepared by incorporating a variety of sterile active ingredients into a sterile vehicle comprising a basic dispersion medium and/or other ingredients. In the case of sterile powders for the preparation of sterile injectable solutions, suspensions or emulsions, the preferred method of preparation is a vacuum drying or lyophilization technique that produces powders of the active ingredient and additional desired optional ingredients from a previously sterile filtered liquid medium. The liquid medium should be adequately buffered if necessary, and the liquid diluent is isotonic prior to injecting sufficient saline or glucose. The preparation of highly concentrated compositions for direct injection is also contemplated, in which case the use of DMSO as a solvent is thought to induce very rapid infiltration and deliver high concentrations of the active agent to a small area.

In certain embodiments, absorption delaying agents may be used in the composition to delay absorption of the injection composition, such as, for example, aluminum monostearate, gelatin, or combinations thereof.

In certain embodiments, the pharmaceutical composition may include one or more pH adjusting agents or buffering agents, including: acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the pharmaceutical composition in an acceptable range.

In certain embodiments, the pharmaceutical composition may comprise one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical composition may be formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

In certain embodiments, the pharmaceutical composition is a liquid. In some embodiments, the composition may be lyophilized and then reconstituted before use.

In certain embodiments, the pharmaceutical composition may include one or more preservatives, for example, to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, the pharmaceutical composition may include one or more antifoaming agents. Antifoaming agents can reduce foaming during processing which can result in coagulation of aqueous dispersions, bubbles in the finished film, or generally impair processing. Exemplary anti-foaming agents include silicon emulsions or sorbitan sesquoleate.

In certain embodiments, the pharmaceutical composition may include one or more antioxidants. Exemplary antioxidants include butylated hydroxytoluene (BHT), sodium ascorbate, ascorbic acid, sodium metabisulfite and tocopherol. In certain embodiments, the one or more antioxidants enhance chemical stability of the composition.

In certain embodiments, the pharmaceutical composition may include one or more stabilizing agents. Exemplary stabilizing agents include, for example: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In certain embodiments, the pharmaceutical composition may include one or more binders. Binders can impart cohesive qualities. Exemplary binders include: alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

In certain embodiments, the pharmaceutical composition may include a carrier or a pharmaceutically-compatible carrier material. These may include any commonly used excipients in pharmaceutics and should be selected on the basis of compatibility with the pharmaceutical compounds described herein. Exemplary carrier materials include binders, suspending agents, disintegration agents, filling agents, surfactants, solubilizers, stabilizers, lubricants, wetting agents, diluents, and the like. Exemplary pharmaceutically-compatible carrier materials may include acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition may include one or more diffusion facilitating agents, dispersing agents, and/or viscosity modulating agents, for example, to control the diffusion and homogeneity of the composition through liquid media or a granulation or blend method. In some embodiments, these agents also facilitate the effectiveness of a coating or eroding matrix. Exemplary diffusion facilitators and dispersing agents may include hydrophilic polymers, electrolytes, Tween®60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (5-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

In certain embodiments the pharmaceutical composition comprises a combination of one or more erosion facilitators with one or more diffusion facilitators.

In certain embodiments, the pharmaceutical composition may include one or more diluents. A diluent is a chemical compound that is used to dilute the substance of interest prior to delivery. Diluents can also be used to stabilize substances because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the pharmaceutical composition may include one or more filling agent. Filling agents may include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the pharmaceutical composition may include one or more lubricants or glidants. These are compounds that prevent, reduce, or inhibit adhesion or friction of materials. Exemplary lubricants may include stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

In certain embodiments, the pharmaceutical composition may include one or more plasticizers. These are compounds used to soften the microencapsulation material or film coatings to make them less brittle. Exemplary plasticizers may include polyethylene glycols such as PEG 300, PEG 400, PEG 600, PEG 1450, PEG 3350, and PEG 800, stearic acid, propylene glycol, oleic acid, triethyl cellulose and triacetin. In some embodiments, the plasticizers may also function as dispersing agents or wetting agents.

In certain embodiments, the pharmaceutical composition may include one or more solubilizers. Exemplary solubilizers may include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In certain embodiments, the pharmaceutical composition may include one or more stabilizers. Exemplary stabilizers may include any antioxidation agents, buffers, acids, preservatives and the like.

In certain embodiments, the pharmaceutical composition may include one or more suspending agents. Exemplary suspending agents may include compounds such as polyvinylpyrrolidone (e.g., polyvinylpyrrolidone K12, polyvinylpyrrolidone K17, polyvinylpyrrolidone K25, or polyvinylpyrrolidone K30), vinyl pyrrolidone/vinyl acetate copolymer (S630), polyethylene glycol (e.g., the polyethylene glycol can have a molecular weight of about 300 to about 6000, or about 3350 to about 4000, or about 7000 to about 5400), sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics (e.g., sodium carboxymethylcellulose, methylcellulose, sodium carboxymethylcellulose, hydroxypropylmethylcellulose, hydroxyethylcellulose), polysorbate-80, sodium alginate, polyethoxylated sorbitan monolaurate, polyethoxylated sorbitan monolaurate, povidone and the like.

In certain embodiments, the pharmaceutical composition may include one or more surfactants. Exemplary surfactants may include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, the surfactants can be included in the pharmaceutical composition to enhance physical stability or for other purposes.

In certain embodiments, the pharmaceutical composition may include one or more viscosity enhancing agents. Exemplary viscosity enhancing agents may include methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

In certain embodiments, the pharmaceutical composition may include one or more wetting agents. Exemplary wetting agents may include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In certain embodiments, the pharmaceutical composition may be manufactured in a conventional manner, such as by means of conventional mixing, dissolving, granulating, dragee-making, levigating, emulsifying, encapsulating, entrapping, or compression processes.

In certain embodiments, the pharmaceutical composition for administration of the TCRs described herein (including cells containing such TCRs and/or polynucleotide sequences encoding such TCRs) may conveniently be presented in unit dosage form and be prepared by any of the methods well known in the art of pharmacy. In general, the pharmaceutical compositions may be prepared by bringing the active ingredient into association with a carrier, and then, if necessary, shaping the product into the desired formulation. In the pharmaceutical composition, the TCRs described herein (or polynucleotides encoding the same) are included in an amount sufficient to produce the desired effect upon the process, condition or disease sought to be treated.

In some embodiments, the pharmaceutical composition may be stored by freezing at a temperature of about 0 °C to about −120 °C, about −10 °C to about −110 °C, about −20 °C to about −100 °C, about −30 °C to about −90 °C, about −40 °C to about −90 °C, about −50 °C to about −90 °C, about −60 °C to about −90 °C, about −65 °C to about −85 °C, or about −70 °C to about −80 °C. In some embodiments, the pharmaceutical composition may be stored by freezing at a temperature of about −60 °C, about −61 °C, about −62 °C, about −63 °C, about −64 °C, about −65 °C, about −66 °C, about −66 °C, about −67 °C, about −68 °C, about −69 °C, about −70 °C, about −71 °C, about −72 °C, about −73 °C, about −74 °C, about −75 °C, about −76 °C, about −77 °C, about −78 °C, about −79 °C, about −80 °C, about −81 °C, about −82 °C, about −83 °C, about −84 °C, about −85 °C, about −86 °C, about −87 °C, about −88 °C, about −89 °C, or about −90 °C. The pharmaceutical composition may be thawed, for example, in a water bath prior to use, avoiding prolonged exposure of the thawed composition to the water bath. The temperature of water bath used to thaw the pharmaceutical composition may be, for example, between about 30 °C to about 44 °C, about 31 °C to about 43 °C, about 32 °C to about 42 °C, about 33 °C to about 41 °C, about 34 °C to about 40 °C, or about 35 °C to about 39 °C. In some embodiments, the temperature of water bath used to thaw the pharmaceutical composition may be about 30 °C, about 31 °C, about 32 °C, about 33 °C, about 34 °C, about 35 °C, about 36 °C, about 37 °C, about 38 °C, about 39 °C, about 40 °C, about 41 °C, about 42 °C, about 43 °C, about 44 °C, or about 45 °C. The thawed composition may be stored for up to about 15 minutes, up to about 30 minutes, up to about 45 minutes, up to about 1 hour, up to about 75 minutes, up to about 90 minutes, up to about 105 minutes, or up to about 2 hours at ambient temperature prior to administration. In some embodiments, the thawed composition will appear as a clear to slightly opalescent, colorless liquid and be substantially free of visible particulates.

### Kits/Articles of Manufacture

Another aspect of the present invention is are kits and articles of manufacture for use with one or more methods described herein. Suitable kits may include a package or container that comprise the TCRs described herein, or polynucleotides encoding such TCRs, or the composition comprising the same. Suitable containers include, for example, bottles, vials, syringes, and test tubes. In some embodiments, the containers are formed from a variety of materials, such as glass or plastic. Suitable articles of manufacture may contain packaging materials. Examples of pharmaceutical packaging materials include blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions may also be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

In some embodiments, the kit contains a vial containing the composition of the present invention. In some such embodiments, the vial comprises, for example, from about 0.1 to about 20 ml of the pharmaceutical composition, from about 0.1 to about 15 ml of the pharmaceutical composition, from about 0.1 to about 10 ml of the pharmaceutical composition, from about 0.1 to about 9 ml of the pharmaceutical composition, from about 0.1 to about 8 ml of the pharmaceutical composition, from about 0.1 to about 7 ml of the pharmaceutical composition, from about 0.1 to about 6 ml of the pharmaceutical composition, from about 0.1 to about 5 ml of the pharmaceutical composition, from about 0.2 to about 5 ml of the pharmaceutical composition, from about 0.2 to about 4 ml of the pharmaceutical composition, from about 0.2 to about 3 ml of the pharmaceutical composition, from about 0.2 to about 2 ml of the pharmaceutical composition, from about 0.2 to about 1 ml of the pharmaceutical composition, from about 0.5 to about 2 ml of the pharmaceutical composition, from about 0.5 to about 1.75 ml of the pharmaceutical composition, from about 0.5 to about 1.5 ml of the pharmaceutical composition, from about 0.5 to about 1.25 ml of the pharmaceutical composition, from about 0.5 to about 1 ml of the pharmaceutical composition, from about 0.75 to about 1.25 ml of the pharmaceutical composition. In other such embodiments, the vial comprises, for example, about 0.5 ml of the pharmaceutical composition, about 0.55 ml of the pharmaceutical composition, about 0.6 ml of the pharmaceutical composition, about 0.65 ml of the pharmaceutical composition, about 0.7 ml of the pharmaceutical composition, about 0.75 ml of the pharmaceutical composition, about 0.8 ml of the pharmaceutical composition, about 0.85 ml of the pharmaceutical composition, about 0.9 ml of the pharmaceutical composition, about 0.95 ml of the pharmaceutical composition, about 1 ml of the pharmaceutical composition, about 1.05 ml of the pharmaceutical composition, about 1.1 ml of the pharmaceutical composition, about 1.15 ml of the pharmaceutical composition, or about 1.2 ml of the pharmaceutical composition.

### Manufacture of a Medicament

The present invention also relates in part to a use of the TCRs described herein, polynucleotides encoding the TCRs described herein, or a composition comprising the same, in the manufacture of a medicament for use in treating a disease or disorder in a subject in need thereof. In certain embodiments, the disease or disorder may be a proliferative disease or disorder, such as cancer. In some embodiments, the disease or disorder is attributed to HPV infection.

In one aspect, the present invention provides a method for producing an engineered cell expressing a TCR of the invention. The method involves culturing a cell and introducing into the cell a nucleic acid encoding the TCR under conditions sufficient for expression of the TCR in the cell. The cell can be any cell suitable for expressing a TCR, such as a mammalian cell, a human cell, an immune cell, or a T cell. The nucleic acid can be introduced into the cell using various methods known in the art, such as viral transduction, electroporation, or lipofection.

In one embodiment, the method involves isolating T cells from a subject, such as a patient with an HPV-associated disease or disorder, and engineering the T cells to express the TCR of the invention. The engineered T cells can then be expanded in vitro and reintroduced into the subject to elicit an immune response against HPV-infected cells. This approach, known as adoptive cell therapy, has shown promise in the treatment of various types of cancer.

The engineered cells expressing the TCRs of the invention can be produced in large quantities using standard cell culture techniques. For example, the cells can be cultured in a bioreactor or a large-scale cell culture system, such as a wave bioreactor or a perfusion bioreactor. The culture conditions, such as temperature, pH, and nutrient composition, can be optimized to maximize cell growth and TCR expression. The engineered cells can be harvested, purified, and formulated into a pharmaceutical composition for administration to a subject in need thereof.

In another embodiment, the method involves introducing the nucleic acid encoding the TCR into a cell in vivo, such as by direct injection into a tumor or by systemic administration using a targeted delivery system. The in vivo expression of the TCR in the subject's cells can lead to the generation of TCR-expressing T cells that can mount an immune response against HPV-infected cells.

The engineered cells expressing the TCRs of the invention can also be used for various in vitro applications, such as studying the mechanisms of HPV infection and the immune response to HPV, or screening for compounds that can modulate the activity of the TCRs or enhance the efficacy of TCR-based therapies.

### Combination Therapies

In certain embodiments, the compositions and methods of the present invention can be combined with at least one additional therapy. Such additional therapies include radiation therapy, surgery (e.g., debulking), chemotherapy, gene therapy, DNA therapy, virus therapy, RNA therapy, immunotherapy, bone marrow transplantation, nanotherapy, monoclonal antibody therapy, or a combination of the aforementioned therapies. The additional therapy may be in the form of an adjuvant or neoadjuvant therapy.

Any suitable agent that may be combined with the TCRs of the present invention, or polynucleotides encoding the same, or a composition comprising the same, may be used. For example, the agent may be a therapeutic agent, such as a chemotherapy agent, an anti-inflammatory agent, an analgesic, a biological response modifier, a vector comprising such agents, or a cell comprising the therapeutic agent or a nucleic acid encoding the same.

In certain embodiments, the additional agent is administered at or near the same location as the composition comprising the TCRs of the present invention, or polynucleotides encoding the same, is administered. In certain other embodiments, the additional agent is administered at a different location, for example, at the opposite side or extremity.

Administration of the additional agent may be simultaneous with the administration of the composition comprising the TCRs (or corresponding polynucleotides encoding such) of the present invention. In certain embodiments, the additional agent is contained in the same formulation as that containing the vector and can be administered with the vector in one unitary dose. In certain other embodiments, the additional agent is not contained in the same formulation but is administered at the same time or within a limited time frame (e.g., a single day, hour, or fraction of an hour) from the administration of the vector.

Alternatively, administration of the additional agent may be sequential in relation to the administration of TCRs of the present invention. Such may be preferred in instances where minimizing adverse reactions is desired. In such embodiments, the additional agent may be administered on a schedule in accordance with approved dosing regimens for that agent. Alternatively, the agent may be administered in accordance with a schedule that serves to better maximize the therapeutic effects of the combination therapy, while minimizing adverse reactions.

In certain embodiments, a blood test, a pap test, or a clinical biopsy may be used to detect HPV-infected cells prior to the administration of the additional agent.

In certain embodiments, the TCRs of the present invention (including cells comprising the same) or polynucleotides encoding the TCRs, may first be administered to a subject for purposes of treating a disease or disorder and then, at a later date, an additional agent may be administered. In one embodiment, the additional agent is administered at a time when, following the administration of the TCRs of the present invention, the disease or condition is deemed relapsed, progressed, or non-responsive to said TCRs.

Anti-inflammatory agents for use in the such combination therapy include: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.

Exemplary analgesics for use in combination therapy include acetaminophen, oxycodone, tramadol or proporxyphene hydrochloride.

Exemplary biological response modifiers suitable for use in combination therapy according to the present invention include, for example, molecules directed against cell surface markers (e.g., CD4, CD5, etc.); cytokine inhibitors, such as the TNF inhibitors (e.g., etanercept (ENBREL®), adalimumab (HUMIRA®), and infliximab (REMICADE®); chemokine inhibitors; cell signaling inhibitors, such as EGFR inhibitors (e.g., Gefinitnib (IRESSA®) and Erlotinib (TARCEVA®)), nucleotide analogs (e.g., Cidofovir), angiogenesis inhibitors, such as Bevacizumab (AVASTIN®), non-steroidal anti-inflammatory compounds (NSAIDs), such as COX-2-selective drugs (e.g., Celexecob (CELEBREX®)), immune checkpoint inhibitors, such as PD-1 inhibitors (e.g., Pembrolizumab (KEYTRUDA®), Nivolumab (OPDIVO®), and Cemiplimab (LIBTAYO®) and PD-L1 inhibitors (e.g., Atezolizumab (TECENTRIQ®), Avelumab (BAVENCIO®), and Durvalumab (IMFINZI®), adhesion molecule inhibitors, and other adjuvant therapies. The biological response modifiers include monoclonal antibodies as well as recombinant forms of molecules. Exemplary disease-modifying anti-rheumatic drugs (DMARDs) include azathioprine, cyclophosphamide, cyclosporine, methotrexate, penicillamine, leflunomide, sulfasalazine, hydroxychloroquine, Gold (oral (auranofin) and intramuscular), and minocycline.

In certain embodiments, the TCRs provided herein are co-delivered and/or co-expressed along with other cytokines. In certain embodiments, the TCRs provided herein are polynucleotides encoding gene-switch polypeptides and a cytokine, or variant or derivative thereof, and methods and systems incorporating the same. Cytokine is a category of small proteins between about 5-20 kDa that are involved in cell signaling. In some instances, cytokines include chemokines, interferons, interleukins, colony-stimulating factors or tumor necrosis factors. In some embodiments, chemokines play a role as a chemoattractant to guide the migration of cells, and is classified into four subfamilies: CXC, CC, CX3C, and XC. Exemplary chemokines include chemokines from the CC subfamily: CCLI, CCL2 (MCP-1), CCL3, CCL4, CCL5 (RANTES), CCL6, CCL7, CCL8, CCL9 (or CCLI0), CCLI1, CCL12, CCL13, CCL14, CCL15, CCL16, CCLI7, CCL18, CCL19, CCL20, CCL21, CCL22, CCL23, CCL24, CCL25, CCL26, CCL27, and CCL28; the CXC subfamily: CXCLI, CXCL2, CXCL3, CXCL4, CXCL5, CXCL6, CXCL7, CXCL8, CXCL9, CXCLI0, CXCL11, CXCL12, CXCL13, CXCL14, CXCL15, CXCL16, and CXCLI7; the XC subfamily: XCLI and XCL2; and the CX3C subfamily CX3CL1.

In some embodiments, the cytokine is a membrane-bound cytokine, which is co-expressed with a chimeric antigen receptor described herein. In some embodiments, one or more methods described herein further comprise administration of a cytokine. In some instances, the cytokine comprises a chemokine, an interferon, an interleukin, a colony-stimulating factor or a tumor necrosis factor.

In some instances, one or more methods described herein further comprise administration of a cytokine selected from a chemokine, an interferon, an interleukin, a colony-stimulating factor or a tumor necrosis factor. In some instances, one or more methods described herein further comprise administration of a cytokine selected from IL2, IL7, IL12, IL15, a fusion of IL-15 and IL-15Rα, IL21, IFNγ or TNF-α.

In certain embodiments, the TCRs provided herein are co-delivered and/or co-expressed along with interferons. Interferons (IFNs) comprise interferon type I (e.g. IFN-α, IFN-β, IFN-ε, IFN-κ, and IFN-ω), interferon type II (e.g. IFN-γ), and interferon type 111. In some embodiments, IFN-α is further classified into about 13 subtypes including IFNAI, IFNA2, IFNA4, IFNA5, IFNA6, IFNA7, IFNA8, IFNAI0, IFNA13, IFNA14, IFNA16, IFNAI 7, and IFNA21. In certain embodiments, the TCRs provided herein are co-delivered and/or co-expressed along with other interleukins. Interleukins are expressed by leukocytes or white blood cells and they promote the development and differentiation of T and B lymphocytes and hematopoietic cells. Exemplary interleukines include IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36. In some embodiments, interleukins are IL-2, IL-12, IL-15, IL-21 or a fusion of IL-15 and IL-15a.

In some aspects, the interleukin can comprise IL-12. Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as p70), and a homodimer of p40 are formed following protein synthesis. IL-12 is the master regulator of the immune system. IL-12 promotes immune response by activating NK cells and T cells.

In some embodiments, the IL-12 is a single chain IL-12 (scIL-12), protease sensitive IL-12, destabilized IL-12, membrane bound IL-12, intercalated IL-12. In some instances, the IL-12 variants are as described in WO2015/095249, WO2016/048903, WO2017/062953.

In some embodiments, an interleukin comprises mbIL-15. In some embodiments, a mbIL-15 is a membrane-bound chimeric IL-15 which can be co-expressed with a modified effector cell described herein. In some embodiments, the mbIL-15 comprises a full-length IL-15 (e.g., a native IL-15 polypeptide) or fragment or variant thereof, fused in frame with a full length IL-15Rα, functional fragment or variant thereof. In some cases, the IL-15 is indirectly linked to the IL-15Ra through a linker. In some instances, the mbIL-15 is as described in Hurton et al., “Tethered IL-15 augments antitumor activity and promotes a stem-cell memory subset in tumor-specific T cells,” PNAS 2016.

In certain embodiments, the TCRs provided herein are co-delivered and/or co-expressed along with tumor necrosis factors. Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-lBBL, OX40L, and TNF-related apoptosis inducing ligand (TRAIL).

In certain embodiments, the TCRs provided herein are co-delivered and/or co-expressed along with colony stimulating factors. Colony-stimulating factors (CSFs) are secreted glycoproteins that interact with receptor proteins on the surface of hemopoietic stem cells, which subsequently modulates cell proliferation and differentiation into specific kind of blood cells. In some instances, a CSF comprises macrophage colony-stimulating factor, granulocyte macrophage colony-stimulating factor (GM-CSF), granulocyte colony-stimulating factor (G-CSF) or promegapoietin.

In particular embodiments, the TCRs provided herein are delivered to, and/or are expressed in a subject, in conjunction with delivery and/or expression of Interleukin-12 cytokine.

In certain embodiments, in conjunction with delivery or expression of the TCRs described herein, expression of IL-12 in a subject is controlled by constitutive or inducible regulation of expression. In a preferred embodiment, in conjunction with HPV vaccine antigen delivery or expression, expression of IL-12 in a subject is controlled by inducible regulation of expression (also referred to as, inducibly regulated expression of IL-12).

In certain embodiments, the TCRs provided herein may be co-delivered and/or co-expressed along with surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the TCRs.

In some embodiments, the additional therapy is administration of a small molecule enzyme inhibitor or anti-metastatic agent. In some embodiments, the additional therapy is the administration of a side effect limiting agent (e.g., an agent intended to lower the incidence and/or severity of side effects of treatment, such as nausea, etc.). In some embodiments, the additional therapy is radiation therapy. In some embodiments, the additional therapy is surgery. In further embodiments, the surgery is debulking surgery. In some embodiments, the additional therapy is a combination of radiation therapy and surgery. In some embodiments, the additional therapy is gamma irradiation.

In certain embodiments, T cell therapy may be administered before, during, after, or in various combinations with additional cancer therapy, such as immune checkpoint therapy. The administration may be made at intervals ranging from simultaneous to minutes to days to weeks. In embodiments where T cell therapy is provided to the patient separately from the additional therapeutic agent, the operator may generally ensure that no significant time has elapsed between each delivery time, so that the two compounds can continue to exert a beneficial combination effect on the patient. Antibody therapy and anti-cancer therapy may be provided to the patient within about 12 to 24 hours or 72 hours of each other, more specifically within about 6 to 12 hours of each other. In some situations, treatment periods are significant over several days (2, 3, 4, 5, 6 or 7 days) to weeks (1, 2, 3, 4, 5, 6, 7 or 8 weeks) between each dose It may be desirable to extend the period.

Various combinations may be used. For the following examples, the TCR is “A” and the additional therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of this embodiment to a patient will follow the general protocol for administration of the compound, given the toxicity of the agents, if present. Thus, in some embodiments, there is a step of monitoring toxicity resulting from the combination therapy.

### Chemotherapy

In some embodiments, the additional therapy is chemotherapy, such as dacarbazine or temozolomide. The additional therapy can be one or more chemotherapeutic agents known in the art.

The term “chemotherapy” refers to the use of drugs to treat cancer. “Chemotherapy agent” refers to a compound or composition administered in the treatment of cancer. These agents or drugs are classified according to their mode of activity within the cell, for example whether they affect the cell cycle and at what stage. Alternatively, the agent can be characterized based on its ability to directly cross-link DNA, insert into DNA, or influence nucleic acid synthesis to induce chromosomal and mitotic mutations.

Exemplary chemotherapy agents that may be administered in combination with the compostions of the present invention include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 1311-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®). Examples of further chemotherapeutic agents include Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycins, dactinomycin, daunorubicin, detorubicin, 6-diazo-5-oxo-F-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

### Radiation Therapy

In certain embodiments, radiation therapy may be used in combination with any of the methods of TCR treatment described herein. “Radiation therapy” refers to treatment for a disease or disorder (typically, cancer) where radioactive energy is used to destroy cells and their division.

Modern radiation therapy systems use relatively high energy beams of radiation from radioactive isotopes or electron beam X-Ray or as 7-rays generators. Radiation therapy includes external beam radiation, intensity modulated radiation therapy (IMRT), focused radiation, and any form of radiosurgery including Gamma Knife, Cyberknife, Linac, and interstitial radiation (e.g. implanted radioactive seeds, GliaSite balloon), and/or with surgery. Other forms of DNA damage factors that may be implemented in radiation therapy include microwave, proton beam irradiation (U.S. Pat. Nos. 5,760,395 and 4,870,287) and ultraviolet irradiation The dose range of X-rays ranges from 50 to 200 rotgens per day for a long period of time (3 to 4 weeks) to 2000 to 6000 lentgens for a single dose. The range of irradiation of radioactive isotopes can vary widely, depending on the half-life of the isotope, the intensity and type of radiation emitted, and the rate of absorption of neoplastic cells.

In certain embodiments, radiation therapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131 and indium-111.

### Immunotherapy

In some embodiments, the subject is also administered an immunotherapeutic agent. Immunotherapy refers to a treatment that uses a subject's immune system to treat cancer, e.g., cancer vaccines, cytokines, use of cancer-specific antibodies, T cell therapy, and dendritic cell therapy. In the context of cancer therapy, immunotherapy generally relies on the use of immune effector cells and molecules to target and destroy cancer cells. In some embodiments, the subject is also administered an immune modulatory protein.

Examples of immune modulatory proteins include, but are not limited to, B lymphocyte chemoattractant (“BLC”), C—C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C—C motif) Ligand 2 (“MCP-1”), Macrophage colony-stimulating factor (“M-CSF”), Monokine induced by gamma interferon (“MIG”), Chemokine (C—C motif) ligand 2 (“MIP-1 alpha”), Chemokine (C—C motif) ligand 4 (“MIP-1 beta”), Macrophase inflammatory protein-1-delta (“MIP-1 delta”), Platelet-derived growth factor subunit B (“PDGF-BB”), Chemokine (C—C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“HMR-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP S”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokme (C—C motif) ligand 27 (“CTACK”), Chemokine (C—X—C motif) ligand 16 (“CXCL16”), C—X—C motif chemokine 5 (“ENA-78”), Chemokine (C—C motif) ligand 26 (“Eotaxin-3”), Granulocyte chemotactic protein 2 (“GCP-2”), GRO, Chemokine (C—C motif) ligand 14 (“HCC-l”), Chemokine (C—C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C—X—C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C—C motif) ligand 20 (“MIP-3 alpha”), C—C motif chemokine 19 (“MIP-3 beta”), Chemokine (C—C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C—C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 RI, IL-13R2, IL-17B, IL-2 Rα, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), FoUistatin, Follicle-stimulating hormone (“FSH”), Chemokine (C—X—C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD F”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL RI”), Transferrin (“TRF”), WIF-lACE-2, Albumin, AMICA, Angiopoietin 4, B-cell activating factor (“BAFF”), Carbohydrate antigen 19-9 (“CA19-9”), CD 163, Clusterin, CRT AM, Chemokine (C—X—C motif) ligand 14 (“CXCL14”), Cystatin C, Decorin (“DCN”), Dickkopf-related protein 3 (“Dkk-3”), Delta-like protein 1 (“DLL1”), Fetuin A, Heparin binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACT’), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTl-inducible-signaling pathway protein 1 (“WISP-1”), and Receptor Activator of Nuclear Factor k B (“RANK”). In certain preferred embodiments, the subject is also administered IFN-gamma (IFN 7). In particularly preferred embodiments, the subject is pretreated with IFNγ, such as with low doses of IFNγ, prior to administering the TCR-modified immune effector cells disclosed herein (e.g., the adoptive immunotherapy compositions disclosed herein comprising the TCR-T cells disclosed herein).

In some embodiments, the immunotherapy can be an immune checkpoint inhibitor. Immune checkpoints amplify signals (e.g., co-stimulatory molecules) or reject signals. Inhibitory immune checkpoints that can be targeted by blocking immune checkpoints include adenosine A2A receptor (A2AR), B7-H3 (also known as CD276), B and T lymphocyte attenuators (BTLA), cytotoxic T lymphocyte associated proteins 4 (CTLA-4, also known as CD152), indoleamine 2,3-deoxygenase (IDO), killer cell immunoglobulin (KIR), lymphocyte activation gene-3 (LAG3), programmed death 1 (PD-1), T cell immunoglobulin domain and mucin domain 3 (TIM-3) and V-domain Ig inhibitors (VISTA) for T cell activation. In particular, immune checkpoint inhibitors target the PD-1 axis and/or CTLA-4.

The immune checkpoint inhibitor can be a drug such as a small molecule that is a recombinant form of a ligand or receptor, or is an antibody, such as a human antibody (e.g., International Patent Publication WO2015016718; Pardoll, Nat Rev Cancer, 12(4): 252-64, 2012; all of the above documents are incorporated herein by reference). Known inhibitors of the immune checkpoint protein or analogs thereof can be used, in particular chimeric, humanized, and human forms of antibodies. As will be appreciated by those skilled in the art, alternative names and/or equivalent names for specific antibodies mentioned in the present invention may be used. These alternative names and/or equivalent names are interchangeable in the context of the present invention. For example, lambrolizumab is also known by alternative names and/or equivalent names MK-3475 and pembrolizumab.

In some embodiments, the PD-1 binding antagonist is a molecule that inhibits the binding of PD-1 to a ligand binding partner. In certain embodiments, the PD-1 ligand binding partner is PDL1 and/or PDL2. In another embodiment, the PDL1 binding antagonist is a molecule that inhibits the binding of PD-1 to a binding partner. In certain embodiments, the PDL1 binding partner is PD-1 and/or B7-1. In another embodiment, the PDL2 binding antagonist is a molecule that inhibits the binding of PDL2 to a binding partner. In certain embodiments, the PDL2 binding partner is PD-1. The antagonist can be an antibody, antigen-binding fragment thereof, immunoconjugate, fusion protein or oligopeptide. Representative antibodies are described in U.S. Pat. Nos. 8,735,553, 8,354,509 and 8,008,449, all of which are incorporated herein by reference. Other PD-1 axis antagonists for use in the methods provided herein are known in the art, such as described in US patent applications US20140294898, US2014022021 and US20110008369.

In some embodiments, the PD-1 binding antagonist is an anti-PD-1 antibody (e.g., human antibody, humanized form of antibody or chimeric form of antibody). In some embodiments, the anti-PD-1 antibody is selected from the group consisting of nivolumab, pembrolizumab and CT-011. In some embodiments, the PD-1 binding antagonist comprises an extracellular or PD-1 binding portion of PDL1 or PDL2 fused to an immunoconjugate. In some embodiments, the PD-1 binding antagonist is AMP-224. MDX-1106-04, MDX-1106, ONO-4538, BMS-936558 and OPDIVO®, also known as nibol rumap is wherein -PD-1 antibody described in WO2006/121168 call. Pembrolizumab, also known as MK-3475, Merck 3475, lambrolizumab, KEYTRUDA® and SCH-900475 is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T lymphocyte associated protein 4 (CTLA-4), also known as CD152. The complete cDNA sequence of human CTLA-4 is registered under Genbank Accession No. L15006. CTLA-4 is present on the surface of T cells and acts as an “off” switch when binding to CD80 or CD86 on the surface of antigen presenting cells. CTLA4 is a member of the immunoglobulin superfamily that is expressed on the surface of helper T cells and transmits inhibitory signals to T cells. CTLA4 is similar to the T cell co-stimulatory protein CD28, and both molecules bind to CD80 and CD86, also called B7-1 and B7-2 on antigen presenting cells, respectively. CTLA4 transmits an inhibitory signal to T cells, while CD28 transmits a stimulus signal. Intracellular CTLA4 is also found in regulatory T cells and may be important for their function. T cell activation via T cell receptor and CD28 increases the expression of CTLA-4, the inhibitory receptor of the B7 molecule.

In some embodiments, the immune checkpoint inhibitor is an anti-CTLA-4 antibody (e.g., human antibody, human form antibody or chimeric form antibody), antigen binding fragment thereof, immunoconjugate, fusion protein or oligopeptide.

Anti-human-CTLA-4 antibodies (or VH and/or VL domains derived therefrom) suitable for use in the present methods can be prepared using methods well known in the art. Alternatively, anti-CTLA-4 antibodies known in the art can be used. For example, U.S. Pat. No. 8,119,129, WO 01/14424, WO 98/42752; WO 00/37504, also known as tremelimumab; formerly tisilimumab), U.S. Pat. No. 6,207,156; See Hurwitz et al. (1998) Proc Natl Acad Sci USA 95(17): 10067-10071]; Camacho et al. (2004) J Clin Oncology 22(145): Abstract No. 2505 (antibody CP-675206)] and Mokyr et al. (1998) Anti-CTLA-4 antibodies disclosed in Cancer Res 58:5301-5304 can be used in the methods disclosed herein. Antibodies that compete with any of the antibodies known in the art for binding to CTLA-4 can also be used. For example, CTLA-4 antibodies in human form are described in international patent applications WO2001014424, WO2000037504 and US patent U.S. Pat. No. 8,017,114.

Examples of anti-CTLA-4 antibodies include ipilimumab (also known as 10D1, MDX-010, MDX-101 and Yervoy®) or antigen-binding fragments and variants thereof (see e.g., WO 01/14424). In other embodiments, the antibody comprises the heavy and light chain CDRs or VR of ipilimumab. Thus, in one embodiment, the antibody comprises CDR1, CDR2 and CDR3 domains of the VH region of ipilimumab and CDR1, CDR2 and CDR3 domains of the VL region of ipilimumab. In another embodiment, the antibody competes and/or competes for binding to the epitope on the same CTLA-4 as the antibody mentioned above, or binds the same epitope on the same CTLA-4 as the antibody mentioned above. In another embodiment, the antibody has at least about 90% variable region amino acid sequence identity with the aforementioned antibody (e.g., at least about 90%, 95% or 99% variable region identity with ipilimumab). Other molecules for modulating CTLA-4 include CTLA-4 ligands and receptors, such as those described in U.S. Pat. Nos. 5,844,905, 5,885,796 and International Patent Applications WO1995001994 and WO1998042752, and immunoconjugates such as those described in U.S. Pat. No. 8,329,867.

### Hormone Therapy

In some embodiments, a therapeutic agent for use in combination with a TCRs for treating the disorders as described above may be a hormonal regulating agent (e.g., hormone therapy), such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogen (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

### Surgery

In some embodiments, the disclosed TCRs (and related constructs embodying the same) are administered conjointly with surgery.

Therapeutic surgery includes resection in which all or part of cancer tissue is physically removed, dissected and/or destroyed, and the treatment, chemotherapy, radiotherapy, hormone therapy, gene therapy, immunotherapy and/or alternative therapy of this embodiment It can be used in conjunction with other therapies. Tumorectomy refers to the physical removal of at least a portion of a tumor. In addition to tumor resection, surgical treatment may include laser surgery, cold surgery, electrosurgery and microscopically controlled surgery. Debulking refers to the reduction of as much of the volume (i.e., bulk) of a tumor without the intention of a complete eradication. Debulking is usually achieved by surgical removal.

Upon incision of some or all cancer cells, tissues or tumors, cavities may form in the body. Treatment can be by perfusion, direct injection, or topical application to the affected area using additional anti-cancer therapies. Such treatment may be, for example, every 1, 2, 3, 4, 5, 6 or 7 days, every 1, 2, 3, 4 or 5 weeks, or every 1, 2, 3, 4, 5, 6, 7, 8, 9, 10, 11 or 12 months. Such treatment can also be achieved in various dosages.

### Other Combination Therapies

It is also contemplated that other agents may be used in combination with certain aspects of the present embodiments to improve the therapeutic efficacy of the treatment. These additional agents include agents that affect up-regulation of cell surface receptors and GAP junctions, cell proliferation inhibitors and differentiators, cell adhesion inhibitors, agents that increase the sensitivity of hyperproliferative cells to apoptosis inducers, or other biological agents. An increase in intercellular signaling due to an increase in the number of GAP junctions will enhance the anti-hyperproliferative effect on adjacent hyperproliferative cell populations.

In other embodiments, cell proliferation inhibition or differentiation agents can be used in combination with certain aspects of this embodiment to improve the anti-hyperproliferative efficacy of the treatment. It is believed that cell adhesion inhibitors can improve the efficacy of this embodiment. Examples of cell adhesion inhibitors include topical adhesion kinase (FAK) inhibitors and lovastatin. Additionally, it is contemplated that other agents, such as antibody c225, that increase the sensitivity of the hyperproliferative cells to apoptosis, may be used in combination with certain aspects of this embodiment to improve therapeutic efficacy.

### Exemplary Embodiments of the Invention

The foregoing description of specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. Some additional exemplary embodiments (“E”) of the invention include, but are not limited to:
E1. A T cell receptor (TCR) generated as a result of HPV vaccine therapy.
E2. The TCR of E1, wherein the TCR is generated from any known HPV vaccines.
E3. The TCR of E2, wherein the TCR is generated from an UPV quadrivalent (types 6, 11, 16, 18) recombinant vaccine.
E4. The TCR of E3, wherein the TCR is generated from Gardasil.
E5. The TCR of E2, wherein the TCR is generated from an HPV 9-valent recombinant vaccine.
E6. The TCR of E5, wherein the TCR is generated from Gardasil 9.
E7. The TCR of E5, wherein the TCR is generated from Cervavac.
E8. The TCR of E2, wherein the TCR is generated from an HPV bivalent (Types 16 and 18) recombinant vaccine.
E9. The TCR of E8, wherein the TCR is generated from Cervarix.
E10. The TCR of E8, wherein the TCR is generated from Cecolin.
E11. The TCR of E8, wherein the TCR is generated from Walrinvax.
E12. The TCR of E2, wherein the TCR is generated from one or more HPV vaccines disclosed in WO 2022/115470 A1.
E13. The TCR of E12, wherein the TCR is generated from an HPV vaccine comprising a nucleic acid sequence that is a functional variant of SEQ ID NO: 81.
E14. The TCR of E13, wherein the TCR is generated from an HPV vaccine comprising a nucleic acid sequence that is at least 80% identical to SEQ ID NO: 81.
E15. The TCR of E14, wherein the TCR is generated from an HPV vaccine comprising a nucleic acid sequence that is at least 85% identical to SEQ ID NO: 81.
E16. The TCR of E15, wherein the TCR is generated from an HPV vaccine comprising a nucleic acid sequence that is at least 90% identical to SEQ ID NO: 81.
E17. The TCR of E16, wherein the TCR is generated from an HPV vaccine comprising a nucleic acid sequence that is at least 95% identical to SEQ ID NO: 81.
E18. The TCR of E17, wherein the TCR is generated from an HPV vaccine comprising a nucleic acid sequence of SEQ ID NO: 81.
E19. The TCR of E12, wherein the TCR comprises an amino acid sequence that is a functional variant of any one of the amino acid sequences of SEQ ID NOs: 1-80.
E20. The TCR of E19, wherein the TCR comprises an amino acid sequence having at least 80% sequence identity with any one of the amino acid sequences of SEQ ID NOs: 1-80.
E21. The TCR of E20, wherein the TCR comprises an amino acid sequence having at least 85% sequence identity with any one of the amino acid sequences of SEQ ID NOs: 1-80.
E22. The TCR of E21, wherein the TCR comprises an amino acid sequence having at least 90% sequence identity with any one of the amino acid sequences of SEQ ID NOs: 1-80.
E23. The TCR of E22, wherein the TCR comprises an amino acid sequence having at least 95% sequence identity with any one of the amino acid sequences of SEQ ID NOs: 1-80.
E24. The TCR of E23, wherein the TCR comprises an amino acid sequence that is any one of the amino acid sequences of SEQ ID NOs: 1-80.
E25. A method of treating cancer associated with HPV infection in a patient, comprising introducing into the patient a TCR generated as a result of HPV vaccine therapy.
E26. The method of E25, wherein the TCR is generated from any known HPV vaccines.
E27. The method of E26, wherein the HPV vaccine is an HPV quadrivalent (types 6, 11, 16, 18) recombinant vaccine.
E28. The method of E27, wherein the HPV vaccine is Gardasil.
E29. The method of E25, wherein the HPV vaccine is an HPV 9-valent recombinant vaccine.
E30. The method of E29, wherein the HPV vaccine is Gardasil 9.
E31. The method of E29, wherein the HPV vaccine is Cervavac.
E32. The method of E26, wherein the HPV vaccine is an HPV bivalent (Types 16 and 18) recombinant vaccine.
E33. The method of E32, wherein the HPV vaccine is Cervarix.
E34. The method of E32, wherein the HPV vaccine is Cecolin.
E35. The method of E32, wherein the HPV vaccine is Walrinvax.
E36. The method of E26, wherein the HPV vaccine is one or more of those disclosed in WO 2022/115470 A1.
E37. The method of E36, wherein the HPV vaccine comprises a nucleic acid sequence that is a functional variant of SEQ ID NO: 81.
E38. The method of E37, wherein the HPV vaccine comprises a nucleic acid sequence at least 80% identical to SEQ ID NO: 81.
E39. The method of E38, wherein the HPV vaccine comprises a nucleic acid sequence at least 85% identical to SEQ ID NO: 81.
E40. The method of E39, wherein the HPV vaccine comprises a nucleic acid sequence at least 90% identical to SEQ ID NO: 81.
E41. The method of E40, wherein the HPV vaccine comprises a nucleic acid sequence at least 95% identical to SEQ ID NO: 81.
E42. The method of E41, wherein the HPV vaccine comprises the nucleic acid sequence of SEQ ID NO: 81.
E43. The method of E36 wherein the HPV vaccine comprises an amino acid sequence that is a functional variant of any one of the amino acid sequences of SEQ ID NOs: 1-80.
E44. The method of E43, wherein the TCR comprises an amino acid sequence having at least 80% sequence identity with any one of the amino acid sequences of SEQ ID NOs: 1-80.
E45. The method of E44, wherein the TCR comprises an amino acid sequence having at least 85% sequence identity with any one of the amino acid sequences of SEQ ID NOs: 1-80.
E46. The method of E45, wherein the TCR comprises an amino acid sequence having at least 90% sequence identity with any one of the amino acid sequences of SEQ ID NOs: 1-80.
E47. The method of E46, wherein the TCR comprises an amino acid sequence having at least 95% sequence identity with any one of the amino acid sequences of SEQ ID NOs: 1-80.
E48. The method of E47, wherein the TCR comprises an amino acid sequence having any one of the amino acid sequences of SEQ ID NOs. 1-80.
E49. The method of E25, wherein the TCR is engineered to enhance or increase recognition of specific antigens on cancer cells associated with HPV infection.
E50. The method of E49, wherein the specific antigens are E6 and E7 proteins of HPV.
E51. The method of E49, wherein the TCR is introduced into the patient using adoptive cell transfer.
E52. The method of E49, wherein the TCR is introduced into the patient using genetic modification of T cells.
E53. The method of E49, wherein the cancer associated with HPV infection is cervical cancer, anal cancer, or head and neck cancer.
E54. A method of preparing a TCR-T cell for use in treating cancer associated with HPV infection in a patient, comprising isolating T cells from a patient who has received an HPV vaccine, stimulating the T cells with HPV antigens, and isolating the HPV-specific TCR-T cells.
E55. The method of E54, wherein the HPV vaccine is any known HPV vaccine.
E56. The method of E55, wherein the HPV vaccine is an HPV quadrivalent (types 6, 11, 16, 18) recombinant vaccine.
E57. The method of E56, wherein the HPV vaccine is Gardasil.
E58. The method of E55, wherein the HPV vaccine is an HPV 9-valent recombinant vaccine.
E59. The method of E58, wherein the HPV vaccine is Gardasil 9.
E60. The method of E58, wherein the HPV vaccine is Cervavac.
E61. The method of E55, wherein the HPV vaccine is an HPV bivalent (Types 16 and 18) recombinant vaccine.
E62. The method of E61, wherein the HPV vaccine is Cervarix.
E63. The method of E61, wherein the HPV vaccine is Cecolin.
E64. The method of E61, wherein the HPV vaccine is Walrinvax.
E65. The method of E55, wherein the HPV vaccine is one or more of those disclosed in WO 2022/115470 A1.
E66. The method of E65, wherein the HPV vaccine comprises a nucleic acid sequence that is a functional variant of SEQ ID NO: 81.
E67. The method of E66, wherein the HPV vaccine comprises a nucleic acid sequence at least 80% identical to SEQ ID NO: 81.
E68. The method of E67, wherein the HPV vaccine comprises a nucleic acid sequence at least 85% identical to SEQ ID NO: 81.
E69. The method of E68, wherein the HPV vaccine comprises a nucleic acid sequence at least 90% identical to SEQ ID NO: 81.
E70. The method of E69, wherein the HPV vaccine comprises a nucleic acid sequence at least 95% identical to SEQ ID NO: 81.
E71. The method of E70, wherein the HPV vaccine comprises the nucleic acid sequence of SEQ ID NO: 81.
E72. The method of E65, wherein the TCR comprises an amino acid sequence that is a functional variant of any one of the amino acid sequences of SEQ ID NOs: 1-80.
E73. The method of E72, wherein the TCR comprises an amino acid sequence having at least 80% sequence identity with any one of the amino acid sequences of SEQ ID NOs: 1-80.
E74. The method of E73, wherein the TCR comprises an amino acid sequence having at least 85% sequence identity with any one of the amino acid sequences of SEQ ID NOs: 1-80.
E75. The method of E74, wherein the TCR comprises an amino acid sequence having at least 90% sequence identity with any one of the amino acid sequences of SEQ ID NOs: 1-80.
E76. The method of E75, wherein the TCR comprises an amino acid sequence having at least 95% sequence identity with any one of the amino acid sequences of SEQ ID NOs. 1-80.
E77. The method of E76, wherein the TCR comprises an amino acid sequence that is any one of the amino acid sequences of SEQ ID NOs. 1-80.
E78. The method of E54, further comprising engineering the isolated TCRs to enhance or increase recognition of specific antigens on cancer cells associated with HPV infection.
E79. The method of E78, wherein the specific antigens are E6 and E7 proteins of HPV.
E80. The method of E78, further comprising introducing the engineered TCRs into a patient with cancer associated with HPV infection.
E81. The method of E78, wherein the cancer associated with HPV infection is cervical cancer, anal cancer, or head and neck cancer.
E82. The method of E78, wherein the TCR is introduced into the patient using adoptive cell transfer.
E83. The method of E78, wherein the TCR is introduced into the patient using genetic modification of T cells.
E84. The method of E78, wherein the patient has failed to respond to standard cancer therapies.
E85. The method of E78, wherein the patient has received an HPV vaccine prior to the introduction of the engineered TCRs.
E86. The method of E78, wherein the introduction of the engineered TCRs results in a reduction in tumor size and improved overall survival of the patient.
E87. The TCR of E1, wherein the TCR recognizes specific antigens on cancer cells associated with HPV infection.
E88. The TCR of E87, wherein the specific antigens are E6 and E7 proteins of HPV.
E89. The TCR of E1, further comprising at least one modification to enhance the TCR's efficacy or specificity.
E90. The TCR of E89, wherein at least one modification enhances the TCR's specificity to at least one HPV epitope.
E91. The TCR of E89, wherein at least one modification increases the TCR's efficacy against HPV infection.
E92. The TCR of E89, wherein the modification is selected from the group consisting of amino acid substitutions, deletions, insertions, and domain swaps.
E93. The TCR of E1, wherein the TCR is linked to an effector molecule.
E94. The TCR of E93, wherein the effector molecule is selected from the group consisting of: a cytokine, a toxin, a radioisotope, a chemotherapeutic agent, an antibody, an antibody fragment, and an antibody-drug conjugate.
E95. An engineered or isolated cell containing any one TCR of claims 1 to 24 and claims 87-94.
E96. The engineered or isolated cell of E95, wherein the cell is a mammalian cell.
E97. The engineered or isolated cell of E96, wherein the cell is a human cell.
E98. The engineered or isolated cell of E95, wherein the cell is an immune cell.
E99. The engineered or isolated cell of E98, wherein the cell is a T cell.
E100. A nucleic acid molecule encoding any one TCR of claims 1 to 24 and claims 87-94.
E101. A vector comprising the nucleic acid molecule of E100.
E102. The vector of E101, wherein the vector is an adenoviral vector.
E103. A composition containing the cell of E95 and a pharmaceutically acceptable excipient.
E104. A composition containing the nucleic acid of E100 and a pharmaceutically acceptable excipient.
E105. A composition containing the vector of E101 and a pharmaceutically acceptable excipient.
E106. A method of treatment comprising administering the composition of any one of claims 103 to 105 to a subject having a disease or disorder associated with HPV.
E107. The method of treatment of E106, wherein the subject has cervical, anal, or head and throat cancer.
E108. The method of treatment of E106, wherein the disease being treated is recurrent respiratory papillomatosis (RRP).
E109. The method of treatment of E106, wherein the treatment involves treating the patient with one or more additional therapies.
E110. The method of treatment of E109, wherein the additional therapy is debulking surgery.
E111. The method of treatment of E109, wherein the additional therapy consists of a chemotherapy agent, an anti-inflammatory agent, an analgesic, a biological response modifier, a vector comprising such agents, or a cell comprising the agent or a nucleic acid encoding the same.
E112. A method for producing an engineered cell expressing any one TCR of claims 1 to 24 and claims 87-94, comprising introducing the vector of any one of claims 101 to 102 into a cell wherein said vector causes expression of said TCR in said cell.
E113. The method of E112, wherein the engineered cell expressing the TCR is a T cell.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims.

### EXAMPLES

The following examples are included to demonstrate preferred embodiments of the invention, in addition to those embodiments disclosed earlier herein. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Generation of TCRs from HPV Quadrivalent Vaccine

T cells are isolated from a patient who received an HPV quadrivalent (Types 6, 11, 16, 18) recombinant vaccine (e.g., Gardasil). The T cells are then stimulated with HPV antigens, and the T cells expressing the HPV-specific TCRs are isolated by density gradient centrifugation, magnetic cell separation, or flow cytometry sorting. The TCR-T cells and/or polynucleotides encoding the HPV-specific TCRs are then engineered using retroviral/lentiviral transduction or TALEN/CRISPR genome editing techniques to enhance or increase recognition of the HPV E6 and E7 proteins. The engineered TCR-T cells or engineered polynucleotides are cultured and then introduced into a patient with cervical, anal, or head and neck cancer associated with HPV infection. The patient shows a significant reduction in tumor size and improved overall survival.

### Example 2: Generation of TCRs from an HPV 9-Valent Vaccine

T cells are isolated from a patient who received an HPV 9-valent recombinant vaccine (e.g., Gardasil 9). The T cells are then stimulated with HPV antigens, and the T cells expressing the HPV-specific TCRs are isolated by density gradient centrifugation, magnetic cell separation, or flow cytometry sorting. The TCR-T cells and/or polynucleotides encoding the HPV-specific TCRs are then engineered using retroviral/lentiviral transduction or TALEN/CRISPR genome editing techniques to enhance or increase recognition of the HPV E6 and E7 proteins. The engineered TCR-T cells or engineered polynucleotides are cultured and then introduced into a patient with cervical, anal, or head and neck cancer associated with HPV infection. The patient shows a significant reduction in tumor size and improved overall survival.

### Example 3: Generation of TCRs from an HPV Bivalent Vaccine

T cells are isolated from a patient who received an HPV bivalent (Types 16 and 18) recombinant vaccine (e.g., Cervarix). The T cells are then stimulated with HPV antigens, and the T cells expressing the HPV-specific TCRs are isolated by density gradient centrifugation, magnetic cell separation, or flow cytometry sorting. The TCR-T cells and/or polynucleotides encoding the HPV-specific TCRs are then engineered using retroviral/lentiviral transduction or TALEN/CRISPR genome editing techniques to enhance or increase recognition of the HPV E6 and E7 proteins. The engineered TCR-T cells or engineered polynucleotides are cultured and then introduced into a patient with cervical, anal, or head and neck cancer associated with HPV infection. The patient shows a significant reduction in tumor size and improved overall survival.

### Example 4: Generation of TCRs from HPV6/11 Vaccines

T cells are isolated from a patient who received one of the HPV vaccines disclosed in WO 2022/115470 A1. For example, T cells may be isolated from a patient who received an HPV vaccine comprising a nucleic acid sequence of SEQ ID NO: 81 or a functional variant thereof. The T cells are then stimulated with HPV antigens, and the T cells expressing the HPV-specific TCRs are isolated by density gradient centrifugation, magnetic cell separation, or flow cytometry sorting. The TCR-T cells and/or polynucleotides encoding the HPV-specific TCRs are then engineered using retroviral/lentiviral transduction or TALEN/CRISPR genome editing techniques to enhance or increase recognition of the HPV E6 and E7 proteins. The engineered TCR-T cells or engineered polynucleotides are cultured and then introduced into a patient with cervical, anal, or head and neck cancer associated with HPV infection. The patient shows a significant reduction in tumor size and improved overall survival.

### Example 5: Combination Therapy with TCRs and Chemotherapy

Using density gradient centrifugation, magnetic cell separation, or flow cytometry sorting techniques, T cells are isolated from a patient with cervical, anal, or head and neck cancer associated with HPV infection who has been previously received an HPV vaccine. TCR sequences specific for the E6 and E7 proteins of HPV are identified and polynucleotides encoding these TCRs are cloned into an adenoviral vector for genetic engineering of T cells, where said vector is used to cause expression of the HPV-specific TCRs in the patient's T cells.

The patient receives a combination of TCR therapy and chemotherapy. The TCR-engineered T cells are infused into the patient, and the patient receives chemotherapy treatment. The combination therapy results in a synergistic effect, with a significant reduction in tumor size and improved overall survival compared to chemotherapy alone.

### Example 6: Combination Therapy with TCRs and Radiation

Using density gradient centrifugation, magnetic cell separation, or flow cytometry sorting techniques, T cells are isolated from a patient with anal, cervical, or head and neck cancer associated with HPV infection who has been previously received an HPV vaccine. TCR sequences specific for the E6 and E7 proteins of HPV are identified and polynucleotides encoding these TCRs are cloned into an adenoviral vector for genetic engineering of T cells, where said vector is used to cause expression of the HPV-specific TCRs in the patient's T cells.

The patient receives a combination of TCR therapy and radiation therapy. The TCR-engineered T cells are infused into the patient, and the patient receives radiation treatment. The combination therapy results in a synergistic effect, with a significant reduction in tumor size and improved overall survival compared to radiation alone.

### Example 7: Combination Therapy with TCRs and Debulking Surgery

Using density gradient centrifugation, magnetic cell separation, or flow cytometry sorting techniques, T cells are isolated from a patient with anal, cervical, or head and neck cancer associated with HPV infection who has been previously received an HPV vaccine. TCR sequences specific for the E6 and E7 proteins of HPV are identified and polynucleotides encoding these TCRs are cloned into an adenoviral vector for genetic engineering of T cells, where said vector is used to cause expression of the HPV-specific TCRs in the patient's T cells.

The patient receives a combination of TCR therapy and one or more debulking surgeries. The TCR-engineered T cells are infused into the patient, either before or after the patient has undergone one or more debulking procedures. The combination therapy results in a synergistic effect, with a significant reduction in tumor size and improved overall survival compared to debulking alone.

The above-mentioned description and examples should not be considered limiting and may be modified in various ways within the scope of the invention as defined by the appended claims.

### Example 8: Assessing TCR Sequences Following Treatment with HPV6/11 Vaccines

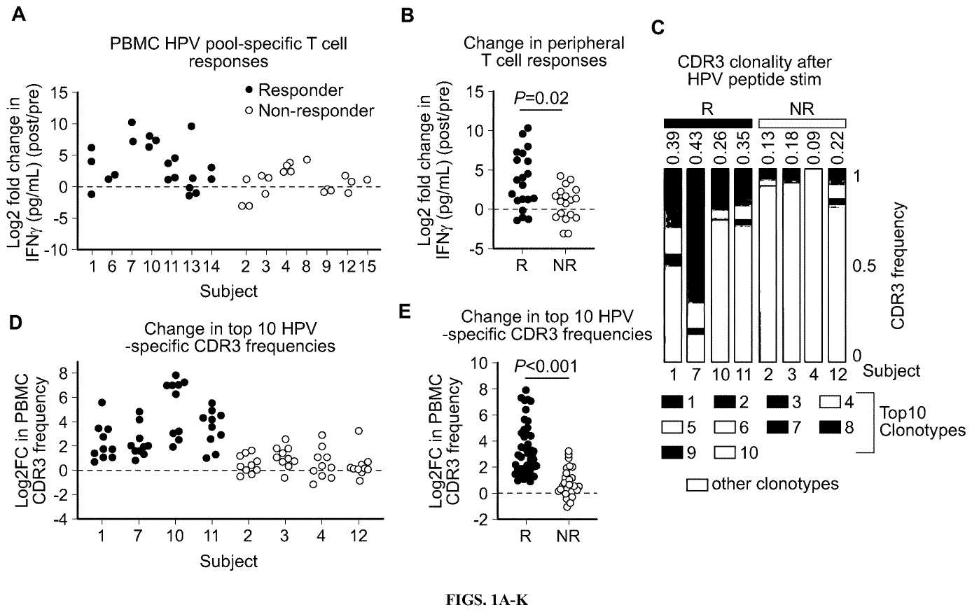

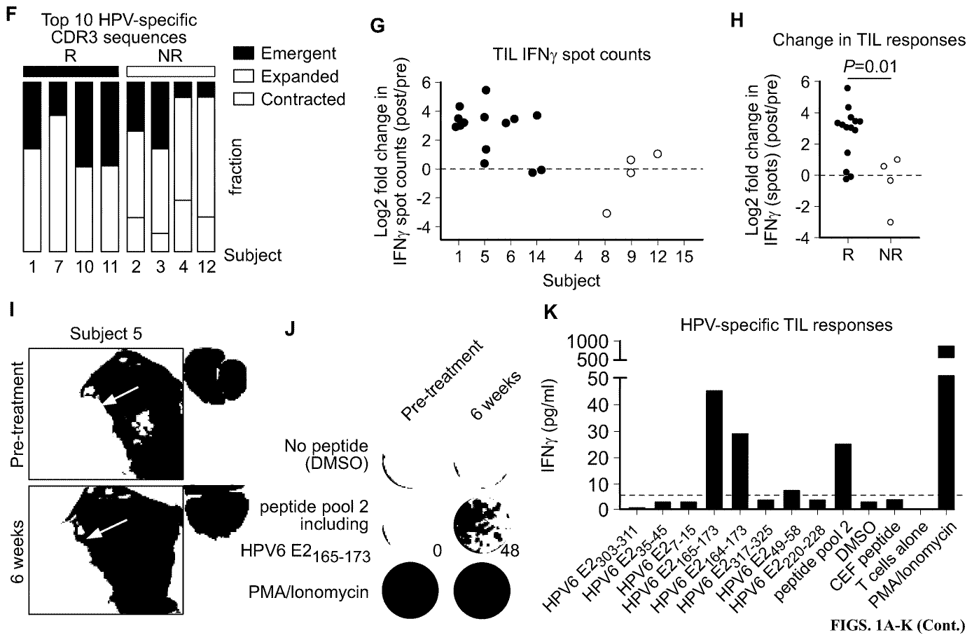

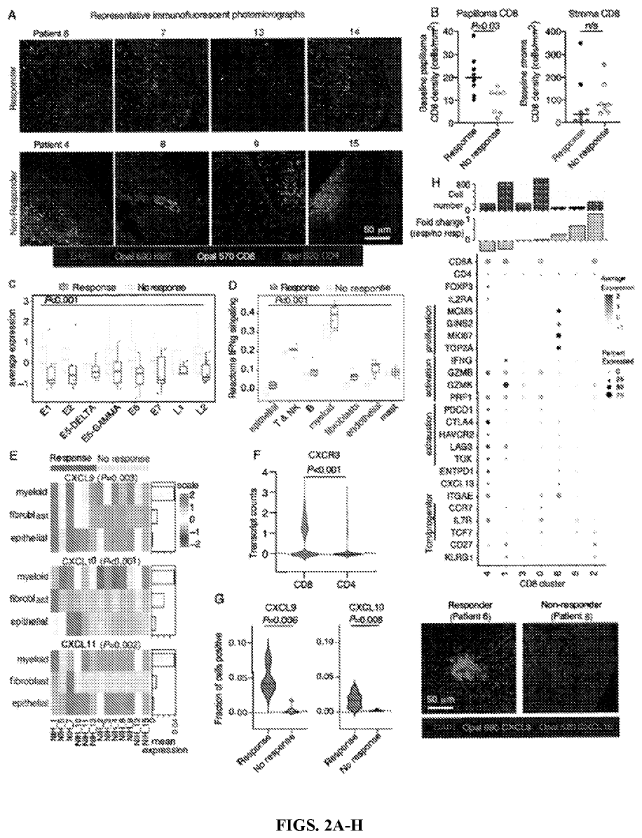

In assessing the clonality of peripheral blood T cell responses to treatment with HPV6/11 vaccines disclosed in WO 2022/115470 A1, the T cell receptor (TCR) CDR3 sequences expressed on HPV-specific T cells were determined using TCRβ sequencing and the FEST assay in subjects with sufficient clinical samples. This approach allows quantification of HPV-specific T cell frequencies in the peripheral blood before and after vaccine treatment. In post-treatment PBMC samples stimulated with HPV 6 and 11 peptides, the top 10 expanded CDR3 sequences determined to be HPV specific accounted for a greater proportion of the TCRβ repertoire in responders compared to non-responders (FIG. 1C) as measured by the Simpson clonality index (P=0.02, unpaired two-tailed t-test). This suggests that HPV-specific T cell clonotypes expand to a greater degree in the blood of patients that develop clinical responses after treatment compared to those that do not. Direct comparison of these HPV-specific CDR3 sequences in unstimulated pre- and post-treatment peripheral T cells revealed consistent expansion in responders (FIG. 2D). Conversely, expansion, no change, or contraction of HPV-specific CDR3 frequencies after treatment compared to before was observed in non-responders. Overall, responders demonstrated a greater magnitude of expansion in peripheral blood HPV-specific CDR3 frequencies compared to non-responders (FIG. 1E, P<0.001, unpaired two-tailed t-test). Considering CDR3 frequencies that are detectable post-treatment and undetectable pre-treatment to be emergent, and CDR3 frequencies detectible pre-treatment but expanded post-treatment to be expanded, both emergent and expanded HPV-specific CDR3 frequencies were detected in all patients analyzed (FIG. 1F).

The above-mentioned descriptions and examples should not be considered limiting and may be modified in various ways within the scope of the invention as defined by the appended claims.

SEQUENCE LISTINGS
SEQ ID NOS. 1-80. TCR CDR3 Amino Acid Sequences
Expressed on HPV-Specific TCells

| | SEQUENCE |
|---|---|
| | ID |
| TCR CDR3 Amino Acid Sequence | NO. |
| CASSTWTQETQYF | 1 |
| CASSPNPAGELFF | 2 |
| CSARARRAGGPGGETQVF | 3 |
| CASSPGQPRATGELFF | 4 |
| CASSVSGGTNEKLFF | 5 |
| CASSQDRAGGTDTQYF | 6 |
| CASSFTEAFF | 7 |
| CATLGAGSHPLGYTF | 8 |
| CASSESWDTEAFF | 9 |
| CASSADRGLAGNTIYF | 10 |
| CASSLNRDSEAFF | 11 |
| CASSQETGISRNTEAFF | 12 |
| CASSLAGAYEQYF | 13 |
| CASSLVGAYEQYF | 14 |
| CASSLVSTGELFF | 15 |
| CASTRAHPDTQVF | 16 |
| CASTAGPYYHQPQHF | 17 |
| CASSLDSLAEAFF | 18 |
| CASSLDGRWEKLFF | 19 |
| CASSLDSGSGTEAFF | 20 |
| CASSLAEGGADYGYTF | 21 |
| CASSFGRTNGELFF | 22 |
| CATSRDPSGRAGETQYF | 23 |
| CASSWMDGLTGRRTTDTQYF | 24 |
| CASSTDKTGQPQHF | 25 |
| CASILPGQSEAFF | 26 |
| CASSLGDRGFSGNTIVF | 27 |
| CATSDRDRGPSNQPQHF | 28 |
| CSARDLLAGVQETQYF | 29 |
| CASSSEASGIYEQYF | 30 |
| CSVLPISSYEQYF | 31 |
| CASSEPRSGNTIYF | 32 |
| CASSISLGEGELFF | 33 |
| CASSLLGDEQYF | 34 |
| CASSLGGYEQYF | 35 |
| CASRRGPDTQYF | 36 |
| CASSSPVQGTGELFF | 37 |
| CASSLRTGTDTQYF | 38 |
| CARLTGELFF | 39 |
| CASNQFSQDSNQPQHF | 40 |
| CASRPGYRYNEQFF | 41 |
| CASSQGQTGGTQKLFF | 42 |
| CASRRLAGGGETQYF | 43 |
| CASSFEQGAGEQYF | 44 |
| CASGTYNEQFF | 45 |
| CASSDPAGDNEQFF | 46 |
| CASRLGGGTGFNQPQHF | 47 |
| CASSFTGSPPNTEAFF | 48 |
| CASSLDTYEQYF | 49 |
| CASSPAGQGGFF | 50 |
| CASEGLAGPYNEQFF | 51 |
| CATSDTGDRRYEQYF | 52 |
| CASSATGVGYTF | 53 |
| CASSLISGTGLTEAFF | 54 |
| CASSDPASGYNEQFF | 55 |
| CASSRVGRPHNEQFF | 56 |
| CASSSSYNEQFF | 57 |
| CASSQVNMGLTSEKLFF | 58 |
| CASSLQAGSTDTQYF | 59 |
| CSVRGGAGLPSGDQYF | 60 |
| CSWVPPWNEQFF | 61 |
| CASSQGWSGGFHEQYF | 62 |
| CASSLTNTGTPSSYNSPLHF | 63 |
| CASSYIALDEKLFF | 64 |
| CASSLEIPGGTGHSTDTQYF | 65 |
| CASSSGEIRENTF | 66 |
| CASSLNLFQPRSEQFF | 67 |
| CASSQQGAVSTEAFF | 68 |
| CASSPNWQDPDGTEAFF | 69 |
| CASRGTGWPGELFF | 70 |
| CATSDPLWAGGLTDTQYF | 71 |
| CASSGQGILGYGYTF | 72 |
| CASSESGWSNQPQHF | 73 |
| CASSPGQGFSYEQYF | 74 |
| CATRTGTYEQYF | 75 |
| CASSPREYQPQHF | 76 |
| CASSIRGGELFF | 77 |
| CASRGGGKPRGEYEQYF | 78 |
| CSAGGGQGTFEQFF | 79 |
| CASSLAGRRNEKLFF | 80 |

SEQUENCE ID NO. 81:
AdV-HPV6/11 antigen DNA open reading frame sequence
ATGGAAGCCATTGCCAAAAGACTCGACGCTTGCGCCGAGCAGCTGC
TGGAGCTGGCCGAAGAGAATAGCACAGACCTCCACAAACACGTCC
TGCATTGGAAGTGCATGAGACATGCCTCCGTGCTCCTGTATAAGG
CCAAGCAGATGGGACTGTCCCACATTGGAATGCAGGTCGTGCCTC
CCCTCAAGGTCAGCGAAGCCAAAGGCCATAACGCTATCGAAATGC
AAATGCACCTGGAAAGCCTCCTGAGGACCGAATACTCCATGGAAC
CCTGGACCCTCCAGGAAACCTCCTACGCTATGTGGCAGACACCCC
CTAAGAGGTGCTTTAAGAAAAGAGGAAAGACAGTGGAAGTGAAAT
TCGATGGCTGTGCCAATAACACAATGGATTACGTCGTGTGGACCG
ATGTGTATGTGCAAGACAATGACACATGGGTCAAGGTCCACTCCA
TGGTGGACGCTAAGGGAATCTATTACACATGCGGACAGTTTAAGA
CATACTATGTGAATTTCGTCAAGGAAGCCGAAAAGTATGGCTCCA
CCAAACACTGGGAGGTCTGCTATGGCTCCACCGTCATCTGTAGCC
CTGCCTCCGTGTCCAGCACAACCCAAGAGGTCAGCATTCCCGAAA
GCACAACCTATACCCCTGCCCAGACCTCCACCCTCGTGTCCAGCT
CCACCAAAGAGGATGCCGTCCAGACACCCCCTAGAAAAAGAGCTA
GAGGAGTGCAACAGTCCCCCTGTAACGCTCTGTGTGTGGCTCACA
TTGGCCCTGTGGATAGCGGAAACCATAACCTCATCACAAACAATC
ACGATCAGCATCAGAGGAGAAATAACTCCAACTCCAGCGCTACCC
CTATCGTCCAGTTTCAGGGAGAGTCCAACTGTCTGAAATGCTTTA
GATATAGACTCAACGATAGACATAGACATCTGTTTGACCTCATCT
CCAGCACATGGCATTGGGCCAGCTCCAAGGCTCCCCATAAGCACG
CCATTGTGACAGTGACATACGATAGCGAAGAGCAAAGACAACAGT
TTCTGGATGTGGTCAAGATTCCCCCTACCATTAGCCATAAGCTCG
GCTTTATGTCCCTGCATCTGCTCCATTGCTATGAGCAACTGGAAG
ACTCCAGCGAAGACGAAGTGGATATGGCAGACGATTCTGCACTGC
ATAAAAAGTACCCATTCCTGAACCTGCTGCATACCCCACCGCATC
GCCCACCGCCACTGTGTCCGCAAGCTCCACGCAAGACCCAATGCA
AGCGCCGTCTGGGTAACGAGCACGAGGAATCCAACTCCCCGCTGG
CTACTCCGTGTACAGCCGAAATCTATGCCTATGCCTATAAGAATC
TGAAAGTGGTCCAATGCACAGAGACAGACATTAGAGAAGTGCAAC
AGCTCCTGCTCGGCACACTGAATATCGTCTGCCCTATCTGTGCCC
CTAAGACAACAGCCGAAATCTATAGCTATGCCTATAAGCAACTGA
AAGTGCTCCAATGCAAGCGCCGTCTGGGTAACGAGCACGAGGAAT
CCAACTCCCCGCTGGCTACTCCGTGTGTTTGGCCGACTCTGGACC
CGTGGACCGTGGAAACTACCACTTCTTCCCTGACTATCACTACCT
CCACCAAGGACGGCACCACTGTTACTGTTCAACTGCGTACCGCTG
AGATTTACGCTTACGCTTACAAAAACCTCAAGGTCGTGATGCACG
GCAGGCACGTCACCCTCAAGGATATCGTCCTGGATCTGCAACCCC
CTGACCCTGTGGGACTGCATTGCTATGAGCAACTGGTGGATTCCA
GCGAAGACGAAGTGGATGAGGTGGACGGACAGGATAGCCAACCCC
TCAAGCAACACTTTCAGATTGTGACATGCTGTCACTGTTACGAAC
AGCTGGAGGATAGCTCCGAGGATGAGGTGGACATGGAAAGCGCTA
ACGCCAGCACAAGCGCTACCACAATCGACCAGCTCTGCAAAACCT
TTAACCTCTCCATGCACACACTGCAAATCAACTGCGTCTTCTGTA
AGAATGCCCTCACCACAGCCGAAATCTATAGCTATGCCTATAAGC
ATCTGAAAGTGCTCTTCAGGGGCGGATACCCTTACGCTGCCTGTG
CCTGTTGCCTGGAGTTTCACGGAAAGATTAACCAATACGCTCACT
TTGACTATGCCGGATACGCTACCACAGTGGAAGAGGAAACCAAAC
AGGATATCCTCGACGTGCTGATTAGATGTTACCTCTGCCATAAGC
CTCAGTGTGAGGTCGAGAAAGTGAAACACATTCTGACAAAGGCTA
GATTTATCAAACTGAATTGCACAAGAAAAGGCAGGTGCCTCCACT
GTTGGACAACCTGTATGGAAGACATGCTGCCT

1. A T cell receptor (TCR) generated as a result of therapy with an HPV vaccine, wherein the HPV vaccine is selected from: an HPV quadrivalent recombinant vaccine; an HPV 9-valent recombinant vaccine; or an HPV bivalent recombinant vaccine.

2. The TCR of claim 1, wherein the HPV vaccine is an HPV bivalent recombinant vaccine.

3. The TCR of claim 2, wherein the HPV vaccine comprises a nucleic acid sequence having at least 80% sequence identity with SEQ ID NO: 81.

4. The TCR of claim 2, wherein the HPV vaccine comprises a nucleic acid sequence having at least 90% sequence identity with SEQ ID NO: 81.

5. The TCR of claim 2, wherein the HPV vaccine comprises a nucleic acid sequence having at least 95% sequence identity with SEQ ID NO: 81.

6. The TCR of claim 2, wherein the HPV vaccine comprises a nucleic acid sequence having at least 97% sequence identity with SEQ ID NO: 81.

7. The TCR of claim 2, wherein the HPV vaccine comprises a nucleic acid sequence having at least 98% sequence identity with SEQ ID NO: 81.

8. The TCR of claim 2, wherein the HPV vaccine comprises a nucleic acid sequence having at least 99% sequence identity with SEQ ID NO: 81.

9. The TCR of claim 2, wherein the HPV vaccine comprises the nucleic acid sequence of SEQ ID NO: 81 or a codon degenerate variant thereof.

10. The TCR of claim 2, wherein the HPV vaccine comprises the nucleic acid sequence of SEQ ID NO: 81.

11. The TCR of claim 1, wherein the HPV vaccine is Gardasil.

12. The TCR of claim 1, wherein the HPV vaccine is Gardasil 9 or Cervavac.

13. The TCR of claim 1, wherein the HPV vaccine is Cervarix, Cecolin, or Walrinvax.

14. The TCR of claim 1, wherein the TCR comprises an amino acid sequence having at least 80% sequence identity with any one of SEQ ID NOs: 1-80.

15. The TCR of claim 1, wherein the TCR comprises an amino acid sequence having at least 90% sequence identity with any one of SEQ ID NOs: 1-80.

16. The TCR of claim 1, wherein the TCR comprises an amino acid sequence having at least 95% sequence identity with any one of SEQ ID NOs: 1-80.

17. The TCR of claim 1, wherein the TCR comprises an amino acid sequence having at least 97% sequence identity with any one of SEQ ID NOs: 1-80.

18. The TCR of claim 1, wherein the TCR comprises an amino acid sequence having at least 98% sequence identity with any one of SEQ ID NOs: 1-80.

19. The TCR of claim 1, wherein the TCR comprises an amino acid sequence having at least 99% sequence identity with any one of SEQ ID NOs: 1-80.

20. The TCR of claim 1, wherein the TCR comprises an amino acid sequence of any one of SEQ ID NOs: 1-80 or a conservatively-substituted variant thereof.

21. The TCR of claim 1, wherein the TCR comprises the amino acid sequence of any one of SEQ ID NOs: 1-80.

22. The TCR of claim 1, wherein the TCR recognizes specific antigens on cancer cells associated with HPV infection.

23. The TCR of claim 22, wherein the specific antigens are E6 and E7 proteins of HPV.

24. The TCR of claim 1, further comprising a modification that enhances the TCR's efficacy against HPV infection.

25. The TCR of claim 1, further comprising a modification that enhances the TCR's specificity to an HPV epitope.

26. The TCR of claim 1, wherein the TCR is linked to an effector molecule.

27. The TCR of claim 26, wherein the effector molecule is selected from the group consisting of: a cytokine, a toxin, a radioisotope, a chemotherapeutic agent, an antibody, an antibody fragment, and an antibody-drug conjugate.

28. A method of treating a disease or disorder associated with HPV infection in a subject in need thereof, the method comprising introducing into the subject the TCR of any one of claims 1-27.

29. The method of claim 28, wherein the TCR is introduced into the patient using adoptive cell transfer.

30. The method of claim 28, wherein the disease or disorder is a cancer.

31. The method of claim 30, wherein the cancer is cervical cancer, anal cancer, or head and neck cancer.

32. The method of claim 28, wherein the disease or disorder is Recurrent Respiratory Papillomatosis.

33. The use of the TCR of any one of claims 1-27 in the preparation of a medicament for the treatment of a disease or disorder associated with HPV infection.

34. A method of preparing a TCR-T cell for use in treating a disease or disorder associated with HPV infection in a subject that has been administered an HPV vaccine, the method comprising isolating T cells from the subject, stimulating the T cells with HPV antigens, and isolating the HPV-specific TCR-T cells, wherein the HPV vaccine is selected from: an HPV quadrivalent recombinant vaccine; an HPV 9-valent recombinant vaccine; an HPV bivalent recombinant vaccine.

35. The method of claim 34, wherein the HPV vaccine comprises a nucleic acid sequence having at least 80% sequence identity with SEQ ID NO: 81.

36. The method of claim 34, wherein the HPV vaccine comprises a nucleic acid sequence having at least 90% sequence identity with SEQ ID NO: 81.

37. The method of claim 34, wherein the HPV vaccine comprises a nucleic acid sequence having at least 95% sequence identity with SEQ ID NO: 81.

38. The method of claim 34, wherein the HPV vaccine comprises a nucleic acid sequence having at least 97% sequence identity with SEQ ID NO: 81.

39. The method of claim 34, wherein the HPV vaccine comprises a nucleic acid sequence having at least 98% sequence identity with SEQ ID NO: 81.

40. The method of claim 34, wherein the HPV vaccine comprises a nucleic acid sequence having at least 99% sequence identity with SEQ ID NO: 81.

41. The method of claim 34, wherein the HPV vaccine comprises the nucleic acid sequence of SEQ ID NO: 81 or a codon degenerate variant thereof.

42. The method of claim 34, wherein the HPV vaccine comprises the nucleic acid sequence of SEQ ID NO: 81.

43. An engineered or isolated cell comprising the TCR of any one of claims 1-27.

44. The engineered or isolated cell of claim 43, wherein the cell is a mammalian cell.

45. The engineered or isolated cell of claim 43, wherein the cell is a human cell.

46. The engineered or isolated cell of claim 43, wherein the cell is an immune cell.

47. The engineered or isolated cell of claim 43, wherein the cell is a T cell.

48. A nucleic acid molecule encoding the TCR of any one of claims 1-27.

49. A vector comprising the nucleic acid of claim 48.

50. The vector of claim 49, wherein the vector is an adenoviral vector.

51. A pharmaceutical composition comprising the engineered or isolated cell of claim 43 and a pharmaceutically acceptable excipient.

52. The pharmaceutical composition of claim 51 for use in the preparation of a medicament for use in treating a disease or disorder associated with HPV infection.

53. A method for producing an engineered cell expressing a TCR comprising introducing the nucleic acid of claim 48 to a cell.

54. The method of claim 53, wherein the cell is a T cell.

55. A kit comprising the TCR of any one of claims 1-27, and instructions for use in treating a disease or disorder associated with HPV infection.

56. A kit comprising the engineered or isolated cell of claim 43, and instructions for use in treating a disease or disorder associated with HPV infection.

57. A kit comprising the nucleic acid molecule of claim 48, and instructions for use in treating a disease or disorder associated with HPV infection.

58. A method of treating a disease or disorder associated with HPV infection in a subject in need thereof, the method comprising administering to the subject the TCR of any one of claims 1-27, in combination with a therapy selected from: chemotherapy, radiation therapy, or checkpoint inhibitor therapy.

59. A method of treating a disease or disorder associated with HPV infection in a subject in need thereof, the method comprising administering to the subject the engineered or isolated cell of claim 43, in combination with a therapy selected from: chemotherapy, radiation therapy, or checkpoint inhibitor therapy.

60. A method of treating a disease or disorder associated with HPV infection in a subject in need thereof, the method comprising administering to the subject the engineered or isolated cell of claim 48, in combination with a therapy selected from: chemotherapy, radiation therapy, or checkpoint inhibitor therapy.

61. A method of detecting HPV infection in a subject, the method comprising contacting a sample from the subject with the TCR of any one of claims 1-27 and detecting binding of the TCR to the sample, wherein binding of the TCR to the sample indicates the presence of HPV infection in the subject.

62. A method of manufacturing the engineered cell comprising a TCR, the method comprising culturing a cell and introducing into the cell a nucleic acid encoding the TCR of any one of claims 1-27 under conditions sufficient for expression of the TCR in the cell.
